SEQUENCE LISTING (12 sequences)

SEQ ID NO: 1 - DNA, 414 bp, synthetic construct
  source 1..414: mol_type = other DNA
atgtacttgggactgaactgtgtattcatagtttttctcttaaaaggtgtccagagtgaagtgaagcttgaggagtctggaggaggcttggtgcaacctggaagatccatgaaactctcctgtgttgcctctggattcactttcactaactactggatgaactgggtccgccagtctccagagaagggccttgagtgggttggtgaaattagattgaaatctaataattatgcaacatattatgcggagtctgtgaaagggaggttcaccatctcaagagatgattccaaaagtagtgtctacctgcaaatgaacaacttaagagctgaagacactggcatttattactgtaccagggactcgcggaatgctatggactactggggtcaaggaacctcagtcaccgtctcctca

SEQ ID NO: 2 - DNA, 393 bp, synthetic construct
  source 1..393: mol_type = other DNA
atggagaaagacacactcctgctatgggtcctgcttctctgggttccaggttccacaggtgacattgtgctgacccaatctccagcttctttggctgtgtctctagggcagagggccaccatctcctgcagagccagcgaaagtgttgataattttggctttagttttatgaactggttccaacagaaaccaggacagccacccaaactcctcatctatgttgcatccaaccaaggatccggggtccctgccaggtttagtggcagtgggtctgggacagacttcagcctcaacatccatcctatggaggaggatgatgctgcaatgtatttctgtcagcaaactaaggaaattcctcggacgttcggtggaggcaccaagctggaagtcaaa

SEQ ID NO: 3 - protein, 138 aa, synthetic construct
MYLGLNCVFIVFLLKGVQSEVKLEESGGGLVQPGRSMKLSCVASGFTFTNYWMNWVRQSPEKGLEWVGEIRLKSNNYATYYAESVKGRFTISRDDSKSSVYLQMNNLRAEDTGIYYCTRDSRNAMDYWGQGTSVTVSS

SEQ ID NO: 4 - protein, 131 aa, synthetic construct
MEKDTLLLWVLLLWVPGSTGDIVLTQSPASLAVSLGQRATISCRASESVDNFGFSFMNWFQQKPGQPPKLLIYVASNQGSGVPARFSGSGSGTDFSLNIHPMEEDDAAMYFCQQTKEIPRTFGGGTKLEVK

SEQ ID NO: 5 - DNA, 426 bp, synthetic construct
  source 1..426: mol_type = other DNA
atgaagttgtggttaaactgggtttttcttttaacacttttacatggtatccagtgtgaggtgaagctggtggaatctggaggaggcttggtacagcctgggggttctctgagactctcctgtgcaacttctgggttcaccttcagtgatttctacatggagtgggtccgccagcctccagggaagagactggagtggattgctgcaagtagaaacaaagctaatgattatacaacagagtacagtgcatctgtgaagggtcggttcatcgtctccagagacacttcccaaagcatcctctaccttcagatgaatgccctgagagctgaggacactgccatttattactgtgcaagagattactacggtagtagctactggtacttcgatgtctggggcgcagggaccacggtcaccgtctcctca

SEQ ID NO: 6 - DNA, 399 bp, synthetic construct
  source 1..399: mol_type = other DNA
atggagtttcagacccaggtactcatgtccctgctgctctgcatgtctggtgcctgtgcagacattgtgatgactcagtctccaactttccttgctgtgacagcaagtaagaaggtcaccattagttgcacggccagtgagagcctttattcaagcaaacacaaggtgcactacttggcttggtaccagaagaaaccagagcaatctcctaaactgctgatatacggggcatccaaccgatacattggggtccctgatcgcttcacaggcagtggatctgggacagatttcactctgaccatcagcagtgtacaggttgaagacctcacacattattactgtgcacagttttacagctatcctctcacgttcggtgctgggaccaagctggagctgaaa

SEQ ID NO: 7 - protein, 142 aa, synthetic construct
MKLWLNWVFLLTLLHGIQCEVKLVESGGGLVQPGGSLRLSCATSGFTFSDFYMEWVRQPPGKRLEWIAASRNKANDYTTEYSASVKGRFIVSRDTSQSILYLQMNALRAEDTAIYYCARDYYGSSYWYFDVWGAGTTVTVSS

SEQ ID NO: 8 - protein, 133 aa, synthetic construct
MEFQTQVLMSLLLCMSGACADIVMTQSPTFLAVTASKKVTISCTASESLYSSKHKVHYLAWYQKKPEQSPKLLIYGASNRYIGVPDRFTGSGSGTDFTLTISSVQVEDLTHYYCAQFYSYPLTFGAGTKLELK

SEQ ID NO: 9 - DNA, 417 bp, synthetic construct
  source 1..417: mol_type = other DNA
atgtacttgggactgaactgtgtattcatagtttttctcttaaaaggtgtccagagtgaagtgaaggttgaggagtctggaggaggcttggtgcaacctggaggatccatgaaactctcctgtgttgcctctggattcactttcactaactactggatgaactgggtccgccagtctccagagaaggggcttgagtgggttgctgaaattagattgaaatctaattattatgcaacacattatgcggagtctgtgaaagggaggttcaccatctcaagagatgattccaatagtagtgtctacctgcaaatgaacaacttaagagctgaagacactggcatttattactgtgccagggggggtgactactatgctatggactactggggtcaaggaacctcagtcaccgtctcctca

SEQ ID NO: 10 - DNA, 393 bp, synthetic construct
  source 1..393: mol_type = other DNA
atggagaaagacacactcctgctatgggtcctgcttctctgggttccaggttccacaggtgacattgtactgacccaatctccagcttctttggctgtgtctctagggcagagggccaccatctcctgcagagccagcgaaagtgttgataattttggcattacttttcttaactggttccaactgaaaccaggacagccacccaaactcctcatctatgctgcatccaaccaaggatccggggtccctgccaggtttagtggcagtgggtctgggacagacttcagcctcaacatccatcctgtggaggaggatgatactgcaatgtatttctgtcagcaaagtaaggaggttccgctcacgttcggtgctgggaccaagctggagctgaaa

SEQ ID NO: 11 - protein, 139 aa, synthetic construct
MYLGLNCVFIVFLLKGVQSEVKVEESGGGLVQPGGSMKLSCVASGFTFTNYWMNWVRQSPEKGLEWVAEIRLKSNYYATHYAESVKGRFTISRDDSNSSVYLQMNNLRAEDTGIYYCARGGDYYAMDYWGQGTSVTVSS

SEQ ID NO: 12 - protein, 131 aa, synthetic construct
MEKDTLLLWVLLLWVPGSTGDIVLTQSPASLAVSLGQRATISCRASESVDNFGITFLNWFQLKPGQPPKLLIYAASNQGSGVPARFSGSGSGTDFSLNIHPVEEDDTAMYFCQQSKEVPLTFGAGTKLELK

(19) United States
(12) Patent Application Publication
(10) Pub. No.: US 2025/0180551 A1
(43) Pub. Date: Jun. 5, 2025

(54) LATERAL FLOW ASSAY FOR THE DIAGNOSIS OF DERMATOMYCOSIS

(71) Applicant: Bach Diagnostics Inc., Irvine, CA (US)

(72) Inventor: Dylan BACH, Irvine, CA (US)

(21) Appl. No.: 18/702,376

(22) PCT Filed: Oct. 26, 2022
(86) PCT No.: PCT/US2022/078688

§ 371 Date: Apr. 18, 2024

Related U.S. Application Data
(60) Provisional application No. 63/271,983, filed on Oct. 26, 2021.

(51) Int. Cl. G01N 33/543; B01L 3/00; C07K 16/14; G01N 1/44; G01N 33/569

(52) U.S. Cl. CPC G01N 33/54388; B01L 3/5023; C07K 16/14; G01N 1/44; G01N 33/56961; B01L 2300/0825; G01N 2333/37

(57) ABSTRACT

Provided are compositions and methods for detecting one or more dermatophyte organisms in a sample using capture antibodies.

### CROSS-REFERENCE TO RELATED APPLICATIONS

This application is a U.S. National Stage Application under 35 U.S.C. § 371 of International Application No. PCT/US2022/078688, filed Oct. 26, 2022, which claims priority to, and the benefit of, U.S. Provisional Application No. 63/271,983, filed Oct. 26, 2021. The contents of the aforementioned patent applications are incorporated herein by reference in their entirety.

### REFERENCE TO AN ELECTRONIC SEQUENCE LISTING

The contents of the electronic sequence listing (BACH_001_01US_SeqList_ST26.xml; Size: 14,643 bytes; and Date of Creation: Apr. 17, 2024) is herein incorporated by reference in its entirety.

### BACKGROUND

Cutaneous dermatomycoses are widespread throughout the world, affecting up to 20% of the world population. The causative agents, dermatophytes, are fungi that invade and multiply within keratinized tissues (e.g. skin, hair, and nails) causing infection. The clinical presentation of the resulting infection varies according to the location of the infection and the specific dermatophyte species causing it.

While dermatophytes are a common cause of nail and skin infections, the biological diagnosis is rarely done in the clinical practice, and dermatologists do not typically perform any sampling to diagnose dermatophytosis. Consequently, many patients, although not infected with a dermatophyte, may be treated with systemic antidermatophyte agents and thus exposed to unnecessary drug-related adverse-events. Further, these agents that are directed to the wrong organism will not adequately treat the infection.

Correctly diagnosing dermatophyte infections is a challenge, with complex requirements for collecting samples for biological diagnosis, poor sensitivity of the tests that are available, and a long turnaround time (dermatophytes grow slowly and culture can take up to 4 weeks). Recently, various PCR-based assays have been developed, but these assays require specific primers and cannot be performed in the clinic.

### SUMMARY

In some aspects, the present disclosure provides compositions and methods for detecting one or more dermatophyte organisms in a sample. In some embodiments, the present disclosure provides lateral flow assays for diagnosing infection from a subject sample. In some embodiments, the present disclosure provides antibodies that differentially or specifically bind to one or more dermatophyte organisms. In some embodiments, the present disclosure provides methods of diagnosing dermatophyte infection using a lateral flow assay or antibody of the disclosure.

In some aspects, the present disclosure provides methods of differentially detecting one or more dermatophyte organisms in a sample, said method comprising: a) collecting a sample from a subject; b) incubating the subject sample in a sample buffer comprising a non-ionic detergent at room temperature; c) applying the sample in the sample buffer to a lateral flow assay, wherein the lateral flow assay comprises: i) a nitrocellulose membrane comprising one or more unconjugated capture antibodies that binds to an dermatophyte antigen; ii) a conjugate pad comprising one or more detection antibodies comprising an antibody-nanoparticle conjugate that binds to a dermatophyte antigen; iii) a control antibody that binds to the unconjugated nanoparticle; d) allowing the lateral flow assay to run for at least 10 minutes; e) reading the test display to determine whether one or more dermatophyte organisms are present in the subject sample.

In some aspects, the present disclosure provides lateral flow assays for the differential detection of one or more dermatophyte organisms comprising: a) a nitrocellulose membrane comprising one or more unconjugated capture antibodies that binds to an dermatophyte antigen; b) a conjugate pad comprising one or more detection antibodies comprising an antibody-nanoparticle conjugate that binds to a dermatophyte antigen; c) a control antibody that binds to the unconjugated nanoparticle.

In some embodiments, the one or more capture antibodies and one or more detection antibodies bind to the same antigen. In some embodiments, the one or more capture antibodies and one or more detection antibodies bind to different antigens on the same dermatophyte organism. In some embodiments, wherein the one or more capture and detection antibodies are different antibodies. In some embodiments, the one or more capture and detection antibodies are the same antibodies. In some embodiments, one or more capture antibodies or one or more detection antibodies comprise a VL amino acid sequence at least 70% identical to any one of SEQ ID NO: 4, SEQ ID NO: 8, or SEQ ID NO: 12.

In some embodiments, the one or more capture antibodies or one or more detection antibodies comprise a VH amino acid sequence at least 70% identical to any one of SEQ ID NO: 3, SEQ ID NO: 7, or SEQ ID NO: 11. In some embodiments, the one or more capture antibodies or one or more detection antibodies comprise VH amino acid sequence pair at least 70% identical to any one of SEQ ID NO: 3, SEQ ID NO: 7, or SEQ ID NO: 11, and VL amino acid sequence at least 70% identical to any one of SEQ ID NO: 4, SEQ ID NO: 8, or SEQ ID NO: 12. In some embodiments, one or more capture antibodies or one or more detection antibodies comprise: a) a VH amino acid sequence at least 70% identical SEQ ID NO: 3 and a VL amino acid sequence at least 70% identical to SEQ ID NO: 4; b) a VH amino acid sequence at least 70% identical to SEQ ID NO: 7 and a VL amino acid sequence at least 70% identical to SEQ ID NO: 8; or c) a VH amino acid sequence at least 70% identical to SEQ ID NO: 11 and a VL amino acid sequence at least 70% identical to SEQ ID NO: 12.

In some embodiments, the one or more detection antibodies is conjugated to colloidal gold or a cellulose nanobead. In some embodiments, the one or more detection antibodies are conjugated to different cellulose nanobeads that provide different read-outs, allowing the detection and differentiation of at least two different dermatophyte organisms. In some embodiments, the one or more capture or one or more detection antibodies do not bind to non-dermatophyte organisms in the subject sample. In some embodiments, the one or more capture or one or more detection antibodies do not bind to a yeast. In some embodiments, the yeast is a Candida.

In some embodiments, the one or more dermatophyte organisms are Trichophyton fungi, Microsporum fungi, or Epidermophyton fungi. In some embodiments, the one or more dermatophyte organisms are Microsporum amazonicum, Microsporum audouinii, Microsporum boullardii, Microsporum canis, Microsporum canis var. distortum, Microsporum cookei, Microsporum distortum, Microsporum duboisii, Microsporum equinum, Microsporum ferrugineum, Microsporum fulvum, Microsporum gallinae, Microsporum gypseum, Microsporum langeronii, Microsporum nanum, Microsporum persicolor, Microsporum praecox, Microsporum ripariae, Microsporum rivalieri, Epidermophyton floccosum, Epidermophyton stockdaleae, Trichophyton benhamiae, Trichophyton bullosum, Trichophyton concentricum, Trichophyton equinum, Trichophyton eriotrephon, Trichophyton erinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, andTrichophyton violaceum. In some embodiments, the dermatophyte organism is Trichophyton benhamiae, Trichophyton bullosum, Trichophyton concentricum, Trichophyton equinum, Trichophyton eriotrephon, Trichophyton erinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, or Trichophyton violaceum. In some embodiments, the one or more dermatophyte organisms are T. rubrum, T. interdigitale, E. floccosum, or M. canis.

In some embodiments, the nitrocellulose membrane is an Ahlstrom 6614 membrane, a CN95 membrane, a CN140 membrane, a FF120 Plus membrane, a FF170 Plus membrane, a 90-CNPH-N-SS40 membrane, a 200CNPH-N-SS60 membrane, a FF80 Plus membrane, a CNPH70 membrane, a CN150 membrane, a 15μ membrane, or an 8μ membrane. In some embodiments, the nitrocellulose membrane is an Ahlstrom 6614 membrane, a 90-CNPH-N CN95 membrane, or a CN140 membrane. In some embodiments, the lateral flow assay uses gold nanoparticles for detection and the nitrocellulose membrane is CN95 or CN140. In some embodiments, the nitrocellulose membrane comprises between about 0.1 mg/mL to about 5 mg/mL of the one or more capture antibodies. In some embodiments, the nitrocellulose membrane comprises about 1.0 mg/mL of the one or more capture antibodies.

In some embodiments, the conjugate pad comprises between about 1 μl/cm to about 20 μl/cm of the one or more detection antibodies. In some embodiments, the conjugate pad comprises about 8 μl/cm of the one or more detection antibodies. In some embodiments, the conjugate pad comprises about 8 μl/cm of an antibody-gold conjugate.

In some embodiments, the lateral flow assay can detect one or more dermatophyte organisms in an extract of between about 20 ng/ml to about 500 ng/mL.

In some embodiments, the lateral flow assay is stable for at least 5 years. In some embodiments, the lateral flow assay is stable for at least 5 years at room temperature. In some embodiments, the lateral flow assay is stable for at least 300 days at room temperature with no significant decrease in sensitivity. In some embodiments, the lateral flow assay is stable for at least five years with refrigeration.

### BRIEF DESCRIPTION OF THE FIGURES

FIG. 1 depicts a representation of an example of a lateral flow assay.

FIG. 2A: Chromogenic comparison of the Periodic Acid-Schiff (PAS) stain versus three chromogenic immunoassays developed using the 17B6 mAb conjugated with two different probing enzymes, horseradish peroxidase (HRP), alkaline phosphatase (AP). The assay that produces brown pigmentation is mAb conjugated with HRP. The assay that produces red pigmentation is mAb conjugated with AP. The assay that produces pink pigmentation is an unconjugated version of the mAb that is then detected with a secondary reagent conjugated to AP, as needed. The unconjugated mAb allows control of the staining protocol to produce the most optimal results using any probing enzyme.

FIG. 2B: Micrographs of nail tissue from the same specimen showing the comparison between T. rubrum structures stained by PAS vs. 17B6 monoclonal antibody IHC stain detected by direct conjugation with the various enzymes or detected indirectly with an AP-conjugated secondary reagent. In all mAb assays, there is chromogenic development of more fungal elements, whereas the PAS stain is unable to produce staining in these areas. (20× magnification)

FIG. 2C: Micrographs of nail tissue infected with C. albicans. Unlike PAS, the HRP mAb stain did not stain the Candida structures. A halo from the counterstain can be seen encapsulating the yeast. This was similarly seen with AP and unconjugated-AP stains. (40× magnification).

FIG. 2D: Microscopy images of nail infected with T. interdigitale. A lower binding affinity was evident with the truncated mAb staining pattern (unconjugated w/AP). Longer fungal structures can be seen traversing the keratin in the original PAS. (20× magnification).

FIG. 3A-C: Results from validation assay of colloidal gold lateral flow assay. The presence or absence of T. rubrum in nail samples were confirmed using the 17B6 antibody.

FIG. 4A-4C: shows the results from optimization of mAb gold conjugates.

FIG. 5A-5B show the binding of the different antibodies to dermatophytes and unrelated organisms.

FIGS. 6A-6B show the experimental set up and results of experiments on the sensitivity of different capture and detection antibody pairs.

FIGS. 7A-7B, FIG. 8, and FIG. 9 show the experimental set up and results of a pilot study detecting T. rubrum in patient samples.

FIGS. 10A-10D show the sensitivity of a lateral flow assay after being stressed at 55° C.

FIG. 11 shows the experimental set up and results of various buffers on the sensitivity of lateral flow assays stressed at 55° C.

FIGS. 12A-12B show the experimental set up and results of the effect of different conjugate pads on the lateral flow assay.

FIG. 13 shows the experimental set up and results of the effect of fungal sample concentration on the development of nonspecific binding in the lateral flow assay.

FIG. 14 shows the results of an experiment to test the sensitivity of the 20:1 and 40:1 CNB conjugates with fungal extracts ranging from 250 ng/ml to 31 ng/mL in 2% tergitol sample buffer.

FIG. 15 shows the results of an experiment testing the effect of different concentrations of tergitol when gold is used as the conjugate.

FIGS. 16A-16C and FIGS. 17A-17B show the results of an experiment testing the effect of various nitrocellulose membranes on the sensitivity of the lateral flow assay.

FIGS. 18A-18B show the results of an experiment testing the effect of different concentrations of test (e.g., capture) antibodies in both CNB and gold conjugate assays.

### DETAILED DESCRIPTION

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

It should be understood that singular forms such as “a,” “an,” and “the” are used throughout this application for convenience, however, except where context or an explicit statement indicates otherwise, the singular forms are intended to include the plural. All numerical ranges should be understood to include each and every numerical point within the numerical range, and should be interpreted as reciting each and every numerical point individually. The endpoints of all ranges directed to the same component or property are inclusive, and intended to be independently combinable.

The term “about” when used in connection with a referenced numeric indication means the referenced numeric indication plus or minus up to 10% of that referenced numeric indication. For example, the language “about 50” covers the range of 45 to 55.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

By the term “specifically binds,” as used herein, is meant a molecule, such as an antibody or a small molecule, which recognizes and binds to another molecule or feature, but does not substantially recognize or bind other molecules or features in a sample.

As used herein, the term “contacting” includes in solution and solid phase, for example contacting a sample with an antibody, for example contacting a sample that contains a polysaccharide of interest such as a polysaccharide associated with a fungal infection.

### Lateral Flow Assay

A lateral flow assay (LFA) is made up of a sample pad, a conjugate pad, a nitrocellulose strip that contains test and control lines, and a wicking pad. Each component overlaps by at least 1-2 mm which enables unimpeded capillary flow of the sample.

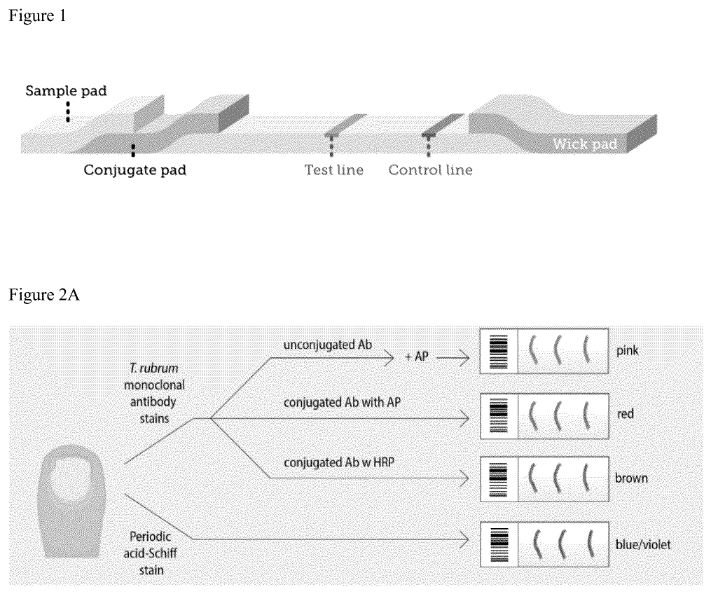

To use the device, a liquid sample such as blood, serum, plasma, urine, saliva, or solubilized solids, is added directly to the sample pad and is wicked through the lateral flow device. The sample pad neutralizes the sample and filters unwanted particulates. The sample can then flow unimpeded to the conjugate pad that contains strongly colored or fluorescent nanoparticles that have an antibody on their surface. When the liquid reaches the conjugate pad, these dried nanoparticles are released and mix with the sample. If there are any target analytes in the sample that the antibody recognizes, these will bind to the antibody. The analyte-bound nanoparticles then flow through a nitrocellulose membrane and across one or more test lines and a control line. The test line (labeled T in FIG. 1) is the primary read-out of the diagnostic and consists of immobilized proteins that can bind the nanoparticle to generate a signal that is correlated to the presence of the analyte in the sample. The fluid continues to flow across the strip until it reaches the control line. The control line contains immobilized proteins that will bind the nanoparticle conjugate with or without the analyte present in solution to confirm that the assay is working properly. After the control line, the fluid flows into the wicking pad which is needed to absorb all of the sample liquid to ensure that there is consistent flow across the test and control lines. In some tests, a chase buffer is applied to the sample port after sample introduction to ensure that all of the sample is transported across the strip. Once all the sample has passed across the test and control lines, the assay is complete and the user can read the results.

The analysis time is dependent on the type of membrane used in the lateral flow assay (larger membranes flow faster but are generally less sensitive) and is typically complete in less than 15 minutes.

The two common assay formats are called “sandwich” and “competitive”. The sandwich assay format is typically used for detecting larger analytes that have at least two binding sites, or epitopes. Usually, an antibody to one binding site is conjugated to the nanoparticle, and an antibody to another binding site is used for the assay's test line. If there is analyte present in the sample, the analyte will bind to both the antibody-nanoparticle conjugate and to the antibody on the test line, yielding a positive signal. The sandwich format results in a signal intensity at the test line that is directly proportional to the amount of analyte present in the sample. Regardless of the quantity of analyte in the sample, an anti-species antibody at the control line will bind the nanoparticle, yielding a strong control line signal that demonstrates that the assay is functioning correctly.

### Samples

Any appropriate sample from a subject may be used in the presently disclosed methods. In some embodiments, the sample is a solid sample comprising for example, nail, nail bed, nail plate, hoof, skin clippings or scrapings. In some embodiments, the sample is an aqueous or liquid sample comprising for example, blood, serum, urine, sweat, or saliva. In some embodiments, the sample is obtained from any area on the subject suspected to harbor a fungal infection. In some embodiments, the sample is obtained from the subject face, foot, toe, skin fold, hand, finger, leg, chest, mouth, or blood. In some embodiments, the subject is a mammal. In some embodiments, the subject is human. In some embodiments, the subject is zoonotic or an agricultural mammal. In some embodiments, the subject is canine, feline, equine, bovine, or porcine.

In some embodiments, the sample does not require any preparation before being incubated in the sample buffer (also termed “extraction buffer”).

Any appropriate amount of sample may be used in the methods and compositions of the instant disclosure. In some embodiments, the sample weighs between about 20 ng to about 500 ng. In some embodiments, the sample weighs about 20 ng, about 25 ng, about 30 ng, about 35 ng, about 40 ng, about 45 ng, about 50 ng, about 55 ng, about 60 ng, about 65 ng, about 70 ng, about 75 ng, about 80 ng, about 85 ng, about 90 ng, about 95 ng, about 100 ng, about 105 ng, about 110 ng, about 115 ng, about 120 ng, about 125 ng, about 130 ng, about 135 ng, about 140 ng, about 145 ng, about 150 ng, about 155 ng, about 160 ng, about 165 ng, about 170 ng, about 175 ng, about 180 ng, about 185 ng, about 190 ng, about 195 ng, about 200 ng, about 205 ng, about 210 ng, about 215 ng, about 220 ng, about 225 ng, about 230 ng, about 235 ng, about 240 ng, about 245 ng, about 250 ng, about 255 ng, about 260 ng, about 265 ng, about 270 ng, about 275 ng, about 280 ng, about 285 ng, about 290 ng, about 295 ng, about 300 ng, about 305 ng, about 310 ng, about 315 ng, about 320 ng, about 325 ng, about 330 ng, about 335 ng, about 340 ng, about 345 ng, about 350 ng, about 355 ng, about 360 ng, about 365 ng, about 370 ng, about 375 ng, about 380 ng, about 385 ng, about 390 ng, about 395 ng, about 400 ng, about 405 ng, about 410 ng, about 415 ng, about 420 ng, about 425 ng, about 430 ng, about 435 ng, about 440 ng, about 445 ng, about 450 ng, about 455 ng, about 460 ng, about 465 ng, about 470 ng, about 475 ng, about 480 ng, about 485 ng, about 490 ng, about 495 ng, or about 500 ng.

### Sample Buffers

Any appropriate sample buffer may be used in the methods and compositions of the present disclosure. In some embodiments, the sample buffer is water; this may be sufficient if there is a high fungal concentration in the sample. In some embodiments, the sample buffer is a comprises a zwitterionic detergent. In some embodiments, the sample buffer comprises a non-ionic detergent. Non-ionic detergents are characterized by their uncharged, hydrophilic headgroups, and typical ones are based on polyoxyethylene or a glycoside. These buffers may solubilize membrane proteins gently, largely preserving their physiological function and structure, by interacting with the hydrophobic membrane regions embedded in the lipid bilayers of the cell membranes.

In some embodiments, the non-ionic sample buffer solubilizes one or more membrane proteins. In some embodiments, the non-ionic detergent is any appropriate sample buffer that may be used to prepare native protein samples, In some embodiments, the non-ionic sample buffer includes, but is not limited to, Tween, Triton, Brij, HEGA, MEGA, NP-40, Triton, tergitol, maltoside, lauryl maltoside, octylthioglucoside, octyl glucoside, digitonin, and CHAPS (3-((3-cholamidopropyl)dimethylammonio)-1-propanesulfonate) detergent buffers. In some embodiments, the sample buffer includes a blocking agent to prevent nonspecific binding in the lateral flow assay. In some embodiments, the sample buffer includes one or more blocking agents. In some embodiments, the blocking agent includes, but is not limited to, a protein blocking agent, Fish gel, bovine serum albumin (BSA), milk proteins, non-fat dry milk (NFDM), whole sera, a polymer (e.g., polyethylene glycol (PEG), polyvinyl alcohol, or polyvinylpyrrolidone). In some embodiments, the blocking agent is a protein blocking agent. In some embodiments, the protein blocking agent is Fish gel.

In some embodiments, the sample buffer and concentration is selected to decrease, reduce, or eliminate nonspecific binding during the assay.

The non-ionic sample buffer may be used at any appropriate concentration. In some embodiments, the buffer has a concentration of about 0.1% to about 10%. In some embodiments, the sample buffer has a concentration of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, or about 10%.

In some embodiments, the sample buffer is about 0.01% to about 10% Tergitol. In some embodiments the sample buffer is about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, or about 10% Tergitol. In some embodiments, the sample buffer is about 2% Tergitol. In some embodiments, the sample buffer is about 0.05% Tergitol.

In some embodiments, the sample buffer is about 0.05% to about 5% lauryl maltoside. In some embodiments the sample buffer is about 0.05%, about 0.10%, about 0.15%, about 0.20%, about 0.25%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% lauryl maltoside. In some embodiments, the sample buffer is about 0.15% lauryl maltoside. In some embodiments, the sample buffer is about 1.5% lauryl maltoside.

In some embodiments, the sample buffer is about 0.05% to about 5% CHAPS. In some embodiments the sample buffer is about 0.05%, about 0.10%, about 0.15%, about 0.20%, about 0.25%, about 0.3%, about 0.4%, about 0.5%, about 0.6%, about 0.7%, about 0.8%, about 0.9%, about 1.0%, about 1.1%, about 1.2%, about 1.3%, about 0.25%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% CHAPS. In some embodiments, the sample buffer is about 0.15% CHAPS.

In some embodiments, the sample buffer is about 0.05% to about 5% Fish Gel. In some embodiments the sample buffer is about 0.05%, about 0.10%, about 0.15%, about 0.20%, about 0.25%, about 0.3%, about 0.4%, about 0.5%, about 0.6%, about 0.7%, about 0.8%, about 0.9%, about 1.0%, about 1.1%, about 1.2%, about 1.3%, about 0.25%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% Fish Gel. In some embodiments, the sample buffer is about 1% Fish Gel.

In some embodiments, the sample buffer is about 0.05% to about 5% digitonin. In some embodiments the sample buffer is about 0.05%, about 0.10%, about 0.15%, about 0.20%, about 0.25%, about 0.3%, about 0.4%, about 0.5%, about 0.6%, about 0.7%, about 0.8%, about 0.9%, about 1.0%, about 1.1%, about 1.2%, about 1.3%, about 0.25%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% digitonin. In some embodiments, the sample buffer is about 1% digitonin.

In some embodiments, the sample buffer is diluted in PBS. In some embodiments, the concentration of PBS is between about 0.1× to about 10× PBS. In some embodiments, the concentration of PBS is about 0.1×, about 0.2×, about 0.3×, about 0.4×, about 0.5×, about 0.6×, about 0.7×, about 0.8×, about 0.9×, about 1.0×, about 1.1×, about 1.2×, about 1.3×, about 1.4×, about 1.5×, about 1.6×, about 1.7×, about 1.8×, about 1.9×, about 2.0×, about 2.1×, about 2.2×, about 2.3×, about 2.4×, about 2.5×, about 2.6×, about 2.7×, about 2.8×, about 2.9×, about 3.0×, about 3.1×, about 3.2×, about 3.3×, about 3.4×, about 3.5×, about 3.6×, about 3.7×, about 3.8×, about 3.9×, about 4.0×, about 4.1×, about 4.2×, about 4.3×, about 4.4×, about 4.5×, about 4.6×, about 4.7×, about 4.8×, about 4.9×, about 5.0×, about 5.1×, about 5.2×, about 5.3×, about 5.4×, about 5.5×, about 5.6×, about 5.7×, about 5.8×, about 5.9×, about 6.0×, about 6.1×, about 6.2×, about 6.3×, about 6.4×, about 6.5×, about 6.6×, about 6.7×, about 6.8×, about 6.9×, about 7.0×, about 7.1×, about 7.2×, about 7.3×, about 7.4×, about 7.5×, about 7.6×, about 7.7×, about 7.8×, about 7.9×, about 8.0×, about 8.1×, about 8.2×, about 8.3×, about 8.4×, about 8.5×, about 8.6×, about 8.7×, about 8.8×, about 8.9×, about 9.0×, about 9.1×, about 9.2×, about 9.3×, about 9.4×, about 9.5×, about 9.6×, about 9.7×, about 9.8×, about 9.9×, or about 10.0× PBS. In some embodiments, the buffer is diluted in 1×PBS.

In some embodiments, the sample buffer is diluted between about .1:10 and about 10:1. In some embodiments, the sample buffer is diluted about 0.1:10, about 0.2:10, about .03:10, about 0.4:10, about 0.5:10, about .06:10, about 0.7:10, about 0.8:10, about 0.9:10, about 1.0:10, about 1.1:10, about 1.2:10, about 1.3:10, about 1.4:10, about 1.5:10, about 1.6:10, about 1.7:10, about 1.8:10, about 1.9:10, about 2.0:10, about 2.1:10, about 2.2:10, about 2.3:10, about 2.4:10, about 2.5:10, about 2.6:10, about 2.7:10, about 2.8:10, about 2.9:10, about 3.0:10, about 3.1:10, about 3.2:10, about 3.3:10, about 3.4:10, about 3.5:10, about 3.6:10, about 3.7:10, about 3.8:10, about 3.9:10, about 4.0:10, about 4.1:10, about 4.2:10, about 4.3:10, about 4.4:10, about 4.5:10, about 4.6:10, about 4.7:10, about 4.8:10, about 4.9:10, about 5.0:10, about 5.1:10, about 5.2:10, about 5.3:10, about 5.4:10, about 5.5:10, about 5.6:10, about 5.7:10, about 5.8:10, about 5.9:10, about 6.0:10, about 6.1:10, about 6.2:10, about 6.3:10, about 6.4:10, about 6.5:10, about 6.6:10, about 6.7:10, about 6.8:10, about 6.9:10, about 7.0:10, about 7.1:10, about 7.2:10, about 7.3:10, about 7.4:10, about 7.5:10, about 7.6:10, about 7.7:10, about 7.8:10, about 7.9:10, about 8.0:10, about 8.1:10, about 8.2:10, about .3:10, about .4:10, about .5:10, about 8.6:10, about 8.7:10, about 8.8:10, about 8.9:10, about 9.0:10, about 9.1:10, about 9.2:10, about 9.3:10, about 9.4:10, about 9.5:10, about .6:10, about 9.7:10, about 9.8:10, about 9.9, or about 1:1. In some embodiments, the sample buffer is diluted in PBS. In some embodiments, the buffer is diluted in 1×PBS. In some embodiments, the sample buffer is diluted in Fish gel. In some embodiments, the sample buffer is diluted in 1% Fish gel. In some embodiments, the sample buffer is diluted in a solution of PBS and Fish Gel. In some embodiments, the sample buffer is diluted in a solution of about 1% Fish gel in about 1× PBS.

In some embodiments, the sample buffer is sufficient to lyse fungal cells in the sample completely. In some embodiments, the sample buffer is sufficient to leave the fungal cells in the sample intact. In some embodiments, the sample buffer does not lyse the fungal cells in the sample, but extracts one or more extracellular components. In some embodiments, the one or more extracellular components is a polypeptide. In some embodiments, the one or more extracellular components is a polysaccharide. In some embodiments, the one or more extracellular components is a carbohydrate. In some embodiments, the sample buffer is sufficient to partially lyse one or more fungal cells in the sample.

In some embodiments, the sample is incubated in the sample buffer for between about 1 minute and 30 minutes, or for between about 1 minute and about 45 minutes, or from about 1 minute and about 60 minutes, or from about 1 minute and about 90 minutes at room temperature. In some embodiments, the sample is incubated in the sample buffer for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, or about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, about 75 minutes, about 76 minutes, about 77 minutes, about 78 minutes, about 79 minutes, about 80 minutes, about 81 minutes, about 82 minutes, about 83 minutes, about 84 minutes, about 85 minutes, about 86 minutes, about 87 minutes, about 88 minutes, about 89 minutes, or about 90 minutes. In some embodiments, the sample and sample buffer are incubated with mixing or stirring.

In some embodiments, after incubation of the sample in the sample buffer, the sample buffer contains between about 0.1 ng/mL to about 10 mg/mL protein. In some embodiments, after incubation of the sample in the sample buffer, the sample buffer contains about 0.1 ng/ml, about 0.2 ng/mL, about 0.3 ng/mL, about 0.4 ng/ml, about 0.5 ng/ml, about 0.6 ng/ml, about 0.7 ng/ml, about 0.8 ng/ml, about 0.9 ng/ml, about 1.0 ng/mL, about 2.0 ng/ml, about 3.0 ng/ml, about 4.0 ng/mL, about 5.0 ng/ml, about 6 ng/mL, about 7.0 ng/ml, about 8.0 ng/ml, about 9.0 ng/ml, about 10 ng/ml, about 20 ng/ml, about 30 ng/ml, about 40 ng/ml, about 50 ng/ml, about 60 ng/ml, about 70 ng/ml, about 80 ng/mL, about 90 ng/ml, about 100 ng/ml, about 200 ng/ml, about 300 ng/mL, about 400 ng/ml, about 500 ng/ml, about 600 ng/ml, about 700 ng/ml, about 800 ng/ml, about 900 ng/ml, about 1.0 mg/mL, about 1.5 mg/mL, about 2.0 mg/mL, about 2.5 mg/mL, about 3.0 mg/mL, about 3.5 mg/mL, about 4.0 mg/mL, about 4.5 mg/mL, about 5.0 mg/mL, about 5.5 mg/mL, about 6.0 mg/mL, about 6.5 mg/mL, about 7.0 mg/mL, about 7.5 mg/mL, about 8.0 mg/mL, about 8.5 mg/mL, about 9.0 mg/mL, about 9.5 mg/mL, or about 10.0 mg/mL.

### Antibodies

In some aspects, the present disclosure provides an antibody that specifically binds one or more dermatophyte organisms. Dermatophytes are filamentous fungi in the genera Trichophyton, Microsporum, and Epidermophyton. These organisms metabolize and subsist on keratin in the skin, hair, and nails.

In some embodiments, the antibody specifically binds one or more of dermatophyte organisms including, but not limited to, Trichophyton fungi, Microsporum fungi, Epidermophyton fungi, Microsporum amazonicum, Microsporum audouinii, Microsporum boullardii, Microsporum canis, Microsporum canis var. distortum, Microsporum cookei, Microsporum distortum, Microsporum duboisii, Microsporum equinum, Microsporum ferrugineum, Microsporum fulvum, Microsporum gallinae, Microsporum gypseum, Microsporum langeronii, Microsporum nanum, Microsporum persicolor, Microsporum praecox, Microsporum ripariae, Microsporum rivalieri, Epidermophyton floccosum, Epidermophyton stockdaleae, Trichophyton benhamiae, Trichophyton bullosum, Trichophyton concentricum, Trichophyton equinum, Trichophyton eriotrephon, Trichophyton erinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, and Trichophyton violaceum. In some embodiments, the dermatophyte organism is Trichophyton benhamiae, Trichophyton bullosum, Trichophyton concentricum, Trichophyton equinum, Trichophyton eriotrephon, Trichophyton erinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, or Trichophyton violaceum. In some embodiments, the dermatophyte organism is T. rubrum, T. interdigitale, E. floccosum, or M. canis.

In some embodiments, the antibody specifically binds between 1 and 5 different dermatophyte species. In some embodiments, the antibody specifically binds between 1 and 5 different dermatophyte species, but does not bind a yeast. In some embodiments, the antibody specifically binds between 1 and 5 different dermatophyte species, but does not bind a Candida yeast. In some embodiments, the antibody specifically binds 1, 2, 3, 4, or 5 different dermatophyte species, but does not bind a yeast (e.g., a Candida yeast). In some embodiments, the antibody differentiates between tinea infections caused by a dermatophyte organism and tinea infections caused by a yeast (e.g. a Candida yeast).

In some embodiments, the antibody specifically binds an intact dermatophyte organism. In some embodiments, the antibody specifically binds a partially degraded dermatophyte organism. In some embodiments, the antibody specifically binds a completely degraded dermatophyte organism.

In some embodiments, the antibody specifically binds one or more cellular components of a dermatophyte organism. In some embodiments, the antibody specifically binds an extracellular component of a dermatophyte organism. In some embodiments, the extracellular component is a lipid, a protein, a polypeptide, a receptor, an extracellular vesicle, a cell-wall component, a chitin, a chitosan, a polysaccharide, a glucan, an adhesin, an agglutinin, a pathogen-associated molecular pattern (PAMP), a glycoprotein, melanin, or fragment or subunit thereof. In some embodiments, the antibody specifically binds one or more components spanning the cell wall and/or membrane, such as a transporter protein or fragment or subunit thereof. In some embodiments, the antibody specifically binds to a component generally found inside the dermatophyte organism, such as DNA, RNA, mRNA, ribosomes, golgi bodies, vesicles, or an organelle component or fragment or subunit thereof.

In some embodiments, the antibody is used as the test antibody (also referred to herein as “capture antibody”). In some embodiments, the antibody is used as the detection antibody. In some embodiments, the antibody is used as both the capture and detection antibodies. In some embodiments, the capture and detection antibodies bind different dermatophyte antigens. In some embodiments, the capture and detection antibodies bind different epitopes of the same dermatophyte antigen. In some embodiments, the pair of capture and detection antibodies is a “self-pair” where the same antibody is used for both capture and detection. In some embodiments, the pair of capture and detection antibodies includes different immunoglobulin antibodies. In some embodiments, the pair of capture and detection antibodies includes different antibodies of the same immunoglobulin class. In some embodiments, the pair of capture and detection antibodies is an IgG-IgM pair. In some embodiments, the pair of capture and detection antibodies is an IgM-IgM pair. In some embodiments, the pair of capture and detection antibodies is an IgG-IgG pair.

In some embodiments, the antibodies described herein are full length antibodies. In some embodiments, the antibodies described herein are selected from a monoclonal antibody, a humanized antibody, a human antibody, a single chain Fv (scFv), a single domain antibody, a Fab, a F (ab′) 2, a single chain diabody, an antibody mimetic, an antibody variable domain, a camelid antibody (also known as VHH or nanobody), a full length antibody, a monospecific antibody, a bispecific antibody, a trispecific antibody, an antigen-binding region, heavy chain, light chain, VHH, VH, VL, a CDR, a variable domain, scFv, Fc, Fv, Fab, F (ab) 2, reduced IgG (rlgG), monospecific Fab2, bispecific Fab2, trispecific Fab3, diabody, bispecific diabody, trispecific triabody, minibody, IgNAR, V-NAR, HclgG, or a combination thereof. In some embodiments, specific residues in the variable domains may be altered to improve binding specificity and/or stability of antibodies and antibody fragments.

In some aspects, the antibody or fragment thereof may be derived from or based on the sequence of an antibody, such as a conventional murine, humanized or human antibody. In some aspects, the antibody or fragment thereof may be derived from or based on an antibody or fragment sequence from a library. In some embodiments, the antibody or fragment thereof derived from or based on the sequence of an antibody or derived from or based on a sequence from a library retains one or more functional activities of the antibody ((e.g., retains at least 80% or more (80%, 85%, 90%, 95%, 97%, 98%, 99% or 100%) of a functional activity). For example, in certain aspects, the antibody or fragment thereof retains one or more of the affinity for antigen and/or selectivity of the antibody or fragment thereof.

In some embodiments the antibody or fragment may comprise a combination of CDR regions from different antibodies that specifically bind to one or more dermatophyte organisms. In some embodiments, the antibody or fragment thereof may comprise a combination of CDR regions from different antibodies that specifically bind to one or more components of a dermatophyte organism as disclosed herein.

In certain aspects, the antibody comprises a light chain portion (VL) comprising the amino acid sequence set forth in any of SEQ ID NO:4, 8, or 12. In some embodiments, the antibody comprises an amino acid sequence at least 80% identical to the any one of SEQ ID NOs: 4, 8, or 12. In some embodiments, the antibody comprises an amino acid sequence at least 80% identical to any one of SEQ ID NOs: 4, 8, or 12 that retains the ability to specifically bind to one or more dermatophyte organisms. In some embodiments, the antibody comprises an amino acid sequence at least 80%-99.9% identical to the any one of SEQ ID NOs: 4, 8, or 12. In some embodiments, the antibody comprises an amino acid sequence at least 80%-99.9% identical to any one of SEQ ID NOs: 4, 8, or 12 that retains the ability to specifically bind to one or more dermatophyte organisms. In some embodiments, the antibody is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identical to the any one of SEQ ID NOs: 4, 8, or 12.

In certain aspects, the antibody comprises a heavy chain portion (VH) comprising the amino acid sequence set forth in any of SEQ ID NO:3, 7, or 11. In some embodiments, the antibody comprises an amino acid sequence at least 80% identical to the any one of SEQ ID NOs: 3, 7, or 11. In some embodiments, the antibody comprises an amino acid sequence at least 80% identical to any one of SEQ ID NOs: 3, 7, or 11 that retains the ability to specifically bind to one or more dermatophyte organisms. In some embodiments, the antibody comprises an amino acid sequence at least 80%-99.9% identical to the any one of SEQ ID NOs: 3, 7, or 11. In some embodiments, the antibody comprises an amino acid sequence at least 80%-99.9% identical to any one of SEQ ID NOs: 3, 7, or 11 that retains the ability to specifically bind to one or more dermatophyte organisms. In some embodiments, the antibody is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identical to the any one of SEQ ID NOs: 3, 7, or 11.

In some embodiments, the antibody comprises a light chain (VL) portion of SEQ ID NO: 4, 7, or 12 and a heavy chain portion (VH) of SEQ ID NO: 3, 7, or 11. In some embodiments, the antibody comprises a light chain (VL) portion of SEQ ID NO: 4 and a heavy chain portion (VH) of SEQ ID NO: 3. In some embodiments, the antibody comprises a light chain (VL) portion of SEQ ID NO: 8 and a heavy chain portion (VH) of SEQ ID NO: 7. In some embodiments, the antibody comprises a light chain (VL) portion of SEQ ID NO: 12 and a heavy chain portion (VH) of SEQ ID NO: 11. The present disclosure also encompasses antibodies and antibody fragments that specifically bind to a dermatophyte organism as disclosed herein, but which have CDR antigen binding site amino acid sequences that are not identical to those sequences disclosed herein. Such antibodies can be preferentially selective for the dermatophyte organism at least 2-fold, at least 5-fold, at least 10-fold, or at least 50-fold higher affinity compared to the present disclosure or antibody fragment thereof. In one aspect, a variant of an antibody or antibody fragment of the present disclosure can be as specific for the dermatophyte organism as a non-variant antibody or antibody fragment of the present disclosure, or can be more specific.

According to certain aspects of the disclosure, variations in antibodies or antibody fragments can occur where they have substantially homologous amino acid sequences, antibodies having substantially similar binding properties, or both. In one aspect of the disclosure, there can be a single amino acid change in the CDR antigen binding sites. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of antibody, such as changing the number or position of glycosylation sites.

Amino acid substitution variants have at least one amino acid residue in the antibody molecule replaced by a different residue. The sites of greatest interest for substitutional mutagenesis include the hypervariable regions or CDRs.

Conservative substitutions involve replacing amino acids with those that have similar charge or hydrophobicity, for example: (1) non-polar: Ala (A), Val (V), Leu (L), Ile (I), Pro (P), Phc (F), Trp (W), Met (M); (2) uncharged polar: Gly (G), Ser(S), Thr (T), Cys (C), Tyr (Y), Asn (N), Gln (Q); (3) acidic: Asp (D), Glu (E); and (4) basic: Lys (K), Arg (R), His (H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phc.

A particularly embodied type of substitutional variant involves substituting one or more hypervariable region residues of a parent antibody (e.g. a humanized or human antibody). Generally, the resulting variant(s) selected for further development will have improved properties relative to the parent antibody from which they are generated.

Several methodologies can be used alone or in combination to improve the stability of an antibody molecule or portion thereof. One methodology that can be used, alone or in combination with one or more of the other methodologies, is engineering the length and/or composition of the linker connecting the scFv domains to stabilize the antibody molecule or portion thereof.

Another potential methodology that can be used, alone or in combination with one or more of the other methodologies described herein, is by introducing at least two amino acid substitutions (also referred to as modifications or mutations) into the VH and/or VL domains of the antibody molecule or portion thereof so as to promote disulfide bond formation (see for example Brinkmann et al., 1993, PNAS, 90:7538-42; Zhu et al., 1997, Prot. Sci. 6:781-8; Reiter et al., 1994, Biochem. 33:5451-9; Reiter et al., 1996, Nature 14:1239-45; Luo et al., 1995, J. Biochem. 118:825-31; Young et al., 1995, FEBS Let. 377:135-9; Glockshuber et al., 1990, Biochem. 29:1362-7).

In certain aspects, one mutation is introduced into each of the VH and VL domains of the antibody molecule or portion thereof to promote interchain disulfide bond formation between the VH and VL domains upon expression of an antibody. In another aspect, the two mutations are introduced in the same domain of the chain. In certain aspect, the two mutations are introduced in different chains. In certain aspects, multiple pairs of two mutations are introduced to promote formation of multiple disulfide bonds. In certain aspects, a cysteine is introduced to promote the disulfide bond formation. A further potential methodology that can be used, alone or in combination with one or more of the other methodologies described herein, is selecting the order of the domains of the antibody molecule or portion thereof. In certain aspects, the orientation of the VH domain relative to the VL domain is optimized for stability.

An additional methodology that can be used, alone or in combination with one or more of the methodologies described herein, is by introducing one or more stabilizing mutations by mutating one or more surface residues of the antibody molecule or portion thereof. In some aspects, one, two, three, four, five, six, or more than six residues are mutated in one or both of the VH and/or VL domain of the antibody molecule or portion thereof. In certain aspects, changes are made in only the VH domain of the antibody molecule or portion thereof. In certain aspects, changes are made in only the VL domain of the antibody molecule or portion thereof. In certain aspects, changes are made in both the VH and VL domains of the antibody molecule or portion thereof. The same number of changes may be made in each domain or a different number of changes may be made in each domain. In certain aspects, one or more of the changes is a conservative amino acid substitution from the residue present in the unmodified, parent antibody molecule or portion thereof. In other aspects, one or more of the changes is a non-conservative amino acid substitution from the residue present in the unmodified, parent antibody molecule or portion thereof. When multiple substitutions are made, either in one or both of the VH or VL domains of the antibody molecule or portion thereof, each substitution is independently a conservative or a non-conservative substitution. In certain aspects, all of the substitutions are conservative substitutions. In certain aspects, all of the substitutions are non-conservative. In certain aspects, at least one of the substitutions is conservative. In certain aspects, at least one or the substitutions is non-conservative.

Yet a further methodology that can be used, alone or in combination with one or more of the additional methodologies described herein, is by introducing one or more substitutions by mutating one or more residues present in the VH and/or VL domain of the antibody molecule or portion thereof to match the most frequent residue at said particular position of a consensus sequence of VH and/or VL domain of known, screened, and/or identified antibodies. In certain aspects, substitutions are introduced at one, two, three, four, five, six, or more than six positions in one or both of the VH domain and/or the VL domain of the antibody molecule or portion thereof. The same number of changes may be made in each domain or a different number of changes may be made in each domain. In certain aspects, one or more of the changes in sequence match that of a given consensus is a conservative amino acid substitution from the residue present in the unmodified VH and/or VL sequence. In other aspects, one or more of the changes represent a non-conservative amino acid substitution from the residue present in the unmodified VH and/or VL sequence. When multiple substitutions are made, either in one or both of the VH or VL domain of the antibody molecule or portion thereof, each substitution is independently a conservative or a non-conservative substitution. In certain aspects, all of the substitutions are conservative substitutions. In certain aspects, all of the substitutions are non-conservative substitutions. In certain aspects, at least one of the substitutions is conservative. In certain aspects, at least one or the substitutions is non-conservative.

### Conjugation of Antibodies

The lateral flow assays of the present disclosure involve the use of detection antibodies that comprise an antibody that binds to antigens from one or more dermatophytes and is conjugated to a detectable marker (e.g. colloidal gold or a cellulose nanosphere). In some embodiments, the detectable marker is a fluorescent marker, a chemoluminescent marker, a surface-enhanced Raman scattering (SERS) marker, an electrochemistry marker, a magnetic marker, a photometric marker, or a colorimetric marker.

In some embodiments, the colorimetric marker is a gold nanoparticle or a cellulose nanobead (CNB).

In addition to their unique color signal, gold nanoparticles also have localized surface plasmon resonance and fluorescence quenching ability, which allow lateral flow assays employing these particles to achieve high sensitivity. In some embodiments, the detectable marker is a gold nanoparticle. In some embodiments, the gold nanoparticle is conjugated to the antibody at a concentration between about 0.5 and about 2.0 OD at 523 nm. In some embodiments, the colloidal gold is used at a concentration of about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 OD at 523. In some embodiments, the colloidal gold is conjugated to the antibody at a concentration of about OD 1.11 at 523. In some embodiments, the colloidal gold is conjugated to the antibody at a concentration of about OD 1.0 at 523.

Cellulose nanobeads may allow for increased sensitivity, faster detection time, and improved reproducibility compared with other detectable markers. In addition, cellulose nanobeads are available in a variety of different colors making them ideal for multiplexing.

In some embodiments, the cellulose nanobeads are conjugated to the detection antibody at a concentration of between 0.01% and about 1.0% solids. In some embodiments, the cellulose nanobeads are conjugated to the detection antibody at a concentration of about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, or about 1% solids. In some embodiments, the cellulose nanobeads are conjugated to the detection antibody at a concentration of about 0.03% solids. In some embodiments, the cellulose nanobeads are conjugated to the detection antibody at a concentration of about 0.06% solids. In some embodiments, the cellulose nanobeads are conjugated to the detection antibody at a concentration of about 0.09% solids. In some embodiments, the cellulose nanobeads are conjugated to the detection antibody at a concentration below about 0.09% solids.

In some embodiments, the cellulose nanobeads are conjugated to the detection antibody at a ratio of about 5:1 to about 100:1 CNB to antibody. In some embodiments, the cellulose nanobeads are conjugated to the detection antibody at a ratio of about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, or about 100:1. In some embodiments, the cellulose nanobeads are conjugated to the detection antibody at a ratio of about 10:1. In some embodiments, the cellulose nanobeads are conjugated to the detection antibody at a ratio of about 20:1. In some embodiments, the cellulose nanobeads are conjugated to the detection antibody at a ratio of about 30:1.

The detectable marker and antibody can be conjugated using any appropriate means known in the art. In some aspects, the process of conjugation should take into account parameters to decrease nonspecific binding and aggregation.

While the conjugation pH is not dependent on the isoelectric point of the specific antibody, the pH for covalent coupling can be very important. In some embodiments, the pH of the conjugation buffer is between about 4 and about 9. In some embodiments, the pH of the conjugation buffer is about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about .5, about 8.6, about 8.7, about 8.8, about 8.9, and about 9.0. In some embodiments, the pH of the conjugation buffer is about pH 6.0, about pH 7.0, about pH 8.0, or about pH 9.0.

Without being bound by theory, because unconjugated gold nanoparticles are not particularly salt stable, they should be maintained in a low-salt environment until they are protected and stabilized by a protein.

In some embodiments, the conjugation buffer may be sodium phosphate buffer, borate buffer, sodium chloride buffer, or a combination thereof. In some embodiments, the conjugation buffer comprises sucrose, a detergent (e.g. Tween 20), trehalose, and/or a stabilizing agent such as PEG.

In some embodiments, the sodium phosphate buffer has a concentration of between about 50 mM and 500 mM. In some embodiments, the sodium phosphate buffer has a concentration of about 50 mM, about 60 mM, about 65 mM, about 70 mM, about 75 mM, about 80 mM, about 85 mM, about 90 mM, about 95 mm, about 100 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM. In some embodiments, the conjugation buffer is 100 mM sodium phosphate buffer. In some embodiments, the conjugation buffer is 100 mM sodium phosphate, pH 6.0. In some embodiments, the conjugation buffer is 100 mM sodium phosphate, pH 7.0.

In some embodiments, the Borate buffer has a concentration of between about 50 mM and 500 mM. In some embodiments, the Borate buffer has a concentration of about 50 mM, about 60 mM, about 65 mM, about 70 mM, about 75 mM, about 80 mM, about 85 mM, about 90 mM, about 95 mm, about 100 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM. In some embodiments, the conjugation buffer is 100 mM Borate buffer. In some embodiments, the conjugation buffer is 100 mM Borate buffer, pH 8.0. In some embodiments, the conjugation buffer is 100 mM Borate buffer, pH 9.0.

In some embodiments, the sodium chloride buffer has a concentration of about 0.5M to about 5M NaCl. In some embodiments, the sodium chloride buffer has a concentration of about 0.5M, about 0.6M, about 0.7M, about 0.8M, about 0.9M, about 1.0M, about 1.1M, about 1.2M, about 1.3M, about 1.4M, about 1.5M, about 1.6M, about 1.7M, about 1.8M, about 1.9M, about 2.0M, about 2.1M, about 2.2M, about 2.3M, about 2.4M, about 2.5M, about 2.6M, about 2.7M, about 2.8M, about 2.9M, about 3.0M, about 3.1M, about 3.2M, about 3.3M, about 3.4M, about 3.5M, about 3.6M, about 3.7M, about 3.8M, about 3.9M, about 4.0M, about 4.1M, about 4.2M, about 4.3M, about 4.4M, about 4.5M, about 4.6M, about 4.7M, about 4.8M, about 4.9M or about 5.0M NaCl. In some embodiments, the conjugation buffer has a concentration of about 1.7M NaCl (10% sodium chloride).

### Nitrocellulose Membranes

The nitrocellulose membrane employed in the lateral flow assays of the present disclosure influence the performance and flow rate of the assay.

In some embodiments, the pore size determines the nitrocellulose membrane employed in the lateral flow assays of the present disclosure. Without wishing to be bound by theory, a nitrocellulose membrane having too small a pore size flows slower, which may increase the development of nonspecific binding in the assay.

Any appropriate nitrocellulose membrane may be used. In some embodiments, the nitrocellulose membrane may be an Ahlstrom 6614 membrane, a CN95 membrane, a CN140 membrane, a FF120 Plus membrane, a FF170 Plus membrane, a 90-CNPH-N-SS40 membrane, a 200CNPH-N-SS60 membrane, a FF80 Plus membrane, a CNPH70 membrane, a CN150 membrane, a 15μ membrane, or an 8μ membrane. In some embodiments, the nitrocellulose membrane is an Ahlstrom 6614 membrane, a 90-CNPH-N CN95 membrane, or a CN140 membrane. In some embodiments, if the lateral flow assay uses gold nanoparticles for detection, the nitrocellulose membrane is CN95 or CN140.

In some embodiments, the nitrocellulose membrane is striped with between about 0.1 mg/mL to about 5 mg/mL test (e.g. capture) antibody. In some embodiments, the nitrocellulose membrane is stiped with about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3.0 mg/mL, about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, about 4.0 mg/mL, about 4.1 mg/mL, about 4.2 mg/mL, about 4.3 mg/mL, about 4.4 mg/mL, about 4.5 mg/mL, about 4.6 mg/mL, about 4.7 mg/mL, about 4.8 mg/mL, about 4.9 mg/mL, or about 5.0 mg/mL test antibody. In some embodiments, the nitrocellulose membrane is striped with about 1.0 mg/mL test antibody.

In some embodiments, the nitrocellulose membrane is striped with between about 0.1 mg/mL to about 5 mg/mL control antibody. In some embodiments, the nitrocellulose membrane is stiped with about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3.0 mg/mL, about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, about 4.0 mg/mL, about 4.1 mg/mL, about 4.2 mg/mL, about 4.3 mg/mL, about 4.4 mg/mL, about 4.5 mg/mL, about 4.6 mg/mL, about 4.7 mg/mL, about 4.8 mg/mL, about 4.9 mg/mL, or about 5.0 mg/mL control antibody. In some embodiments, the nitrocellulose membrane is striped with about 2.0 mg/mL control antibody. In some embodiments, the control antibody is a goat-anti-mouse IgG antibody.

In some embodiments, the conjugate pad is sprayed with between about 1 μl/cm to about 20 μl/cm antibody conjugate (e.g. antibody conjugated to a detectable marker; e.g. “detection antibody”). In some embodiments, the conjugate pad is sprayed with about 1 μl/cm, about 1.1 μl/cm, about 1.2 μl/cm, about 1.3 μl/cm, about 1.4 μl/cm, about 1.5 μl/cm, about 1.6 μl/cm, about 1.7 μl/cm, about 1.8 μl/cm, about 1.9 μl/cm, about 2.0 μl/cm, about 2.1 μl/cm, about 2.2 μl/cm, about 2.3 μl/cm, about 2.4 μl/cm, about 2.5 μl/cm, about 2.6 μl/cm, about 2.7 μl/cm, about 2.8 μl/cm, about 2.9 μl/cm, about 3.0 μl/cm, about 3.1 μl/cm, about 3.2 μl/cm, about 3.3 μl/cm, about 3.4 μl/cm, about 3.5 μl/cm, about 3.6 μl/cm, about 3.7 μl/cm, about 3.8 μl/cm, about 3.9 μl/cm, about 4.0 μl/cm, about 4.1 μl/cm, about 4.2 μl/cm, about 4.3 μl/cm, about 4.4 μl/cm, about 4.5 μl/cm, about 4.6 μl/cm, about 4.7 μl/cm, about 4.8 μl/cm, about 4.9 μl/cm, about 5.0 μl/cm, about 5.5 μl/cm, about 6.0 μl/cm, about 6.5 μl/cm, about 7.0 μl/cm, about 7.5 μl/cm, about 8.0 μl/cm, about 8.5 μl/cm, about 9.0 μl/cm, about 9.5 μl/cm, about 10.0 μl/cm, about 10.5 μl/cm, about 11.0 μl/cm, about 11.5 μl/cm, about 12.0 μl/cm, about 12.5 μl/cm, about 13.0 μl/cm, about 13.5 μl/cm, about 14.0 μl/cm, about 14.5 μl/cm, about 15.0 μl/cm, about 15.5 μl/cm, about 16.0 μl/cm, about 16.5 μl/cm, about 17.0 μl/cm, about 17.5 μl/cm, about 18.0 μl/cm, about 18.5 μl/cm, about 19.0 μl/cm, about 19.5 μl/cm, or about 20 μl/cm antibody conjugate. In some embodiments, the conjugate pad is sprayed with about 8 μl/cm antibody-gold conjugate.

The shelf-life of the lateral flow assays may play an important role in the integrity and sensitivity of the assay. In some embodiments, the lateral flow assays of the present disclosure may be stored for at least one year at room temperature. In some embodiments, the lateral flow assays of the present disclosure may be stored for at least about 100 days to at least 5 years at room temperature with no significant decrease in sensitivity. In some embodiments, the lateral flow assays of the present disclosure may be stored for at least 300 days at room temperature with no significant decrease in sensitivity. In some embodiments, the lateral flow assays of the present disclosure may be stored for at least five years, at least 4 years, at least 3 years, at least two years, or at least 1 year with refrigeration.

### Lateral Flow Assay Methods

In some embodiments, the lateral flow assays of the present disclosure can detect one or more dermatophyte organisms in a fungal extract of at least 31 ng/mL. In some embodiments, the lateral flow assay of the present disclosure can detect one or more dermatophyte organisms in a fungal extract of about 30 ng/mL to about 60 ng/mL, or from about 30 ng/ml to about 80 ng/ml, or from about 40 ng/mL to about 100 ng/ml, or from about 50 ng/ml to about 200 ng/ml, or from about 100 ng/ml to about 400 ng/ml, or from about 400 ng/ml to about 500 ng/ml. In some embodiments, the concentration of one or more dermatophyte organisms that can be detected using a lateral flow assay of the present disclosure varies depending on whether the fungal extract is directly prepared from a subject sample (e.g. a nail or tissue sample) or from a lab cultured fungi.

In some embodiments, the lateral flow assays of the present disclosure can detect one or more dermatophyte organisms in a fungal extract of about 20 ng/mL to about 500 ng/ml. In some embodiments, the lateral flow assays of the present disclosure can detect one or more dermatophyte organisms in a fungal extract of about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/mL, about 70 ng/ml, about 75 ng/ml, about 80 ng/ml, about 85 ng/mL, about 90 ng/ml, about 95 ng/ml, about 100 ng/ml, about 110 ng/ml, about 120 ng/ml, about 130 ng/mL, about 140 ng/ml, about 150 ng/ml, about 160 ng/ml, about 170 ng/ml, about 180 ng/ml, about 190 ng/ml, about 200 ng/ml, about 210 ng/ml, about 220 ng/mL, about 230 ng/ml, about 240 ng/ml, about 250 ng/ml, about 260 ng/ml, about 270 ng/ml, about 280 ng/ml, about 290 ng/ml, about 300 ng/ml, about 310 ng/ml, about 320 ng/ml, about 330 ng/ml, about 340 ng/mL, about 350 ng/mL, about 360 ng/ml, about 370 ng/mL, about 380 ng/ml, about 390 ng/ml, about 400 ng/ml, about 410 ng/ml, about 420 ng/mL, about 430 ng/ml, about 440 ng/ml, about 450 ng/ml, about 460 ng/ml, about 470 ng/mL, about 480 ng/ml, about 490 ng/mL, or about 500 ng/mL.

In some embodiments, between about 10 μl and about 500 μl of the sample in the sample buffer are added to the lateral flow assay. In some embodiments, about 10 μl, about 1lul, about 12 μl, about 13 μl, about 14 μl, about 15 μl, about 16 μl, about 17 μl, about 18 μl, about 19 μl, about 20 μl, about 21 μl, about 22 μl, about 23 μl, about 24 μl, about 25 μl, about 26 μl, about 27 μl, about 28 μl, about 29 μl, about 30 μl, about 31 μl, about 32 μl, about 33 μl, about 34 μl, about 35 μl, about 36 μl, about 37 μl, about 38 μl, about 39 μl, about 40 μl, about 41 μl, about 42 μl, about 43 μl, about 44 μl, about 45 μl, about 46 μl, about 47 μl, about 48 μl, about 49 μl, about 50 μl, about 55 μl, about 60 μl, about 65 μl, about 70 μl, about 75 μl, about 80 μl, about 85 μl, about 90 μl, about 95 μl, about 100 μl, about 110 μl, about 120 μl, about 130 μl, about 140 μl, about 150 μl, about 160 μl, about 170 μl, about 180 μl, about 190 μl, about 200 μl, about 210 μl, about 220 μl, about 230 μl, about 240 μl, about 250 μl, about 260 μl, about 270 μl, about 280 μl, about 290 μl, about 300 μl, about 310 μl, about 320 μl, about 330 μl, about 340 μl, about 350 μl, about 360 μl, about 370 μl, about 380 μl, about 390 μl, about 400 μl, about 410 μl, about 420 μl, about 430 μl, about 440 μl, about 450 μl, about 460 μl, about 470 μl, about 480 μl, about 490 μl, or about 500 μl of the sample in the sample buffer are added to the lateral flow assay. In some embodiments, between about 50 μl-150 μl of the sample in the sample buffer are added to the lateral flow assay. In some embodiments, about 100 μl of the sample in the sample buffer are added to the lateral flow assay.

In some embodiments, the assay is run by 1) collecting a sample from a subject; 2) incubating the sample in a sample buffer; 3) applying the sample buffer containing the sample to the sample pad of the lateral flow assay (see FIG. 1); 4) allowing the test to “run” for a determined period of time; and 5) detecting test antibody and control antibody binding.

In some embodiments, the lateral flow assay runs for between 5 and 90 minutes. In some embodiments, the lateral flow assay runs for about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, or about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, about 75 minutes, about 76 minutes, about 77 minutes, about 78 minutes, about 79 minutes, about 80 minutes, about 81 minutes, about 82 minutes, about 83 minutes, about 84 minutes, about 85 minutes, about 86 minutes, about 87 minutes, about 88 minutes, about 89 minutes, or about 90 minutes. In some embodiments, the lateral flow assay runs for about 15 minutes. In some embodiments, the lateral flow assay runs for no more than 30 minutes. In some embodiments, the lateral flow assay runs at a temperature between about 20° C. and about 25° C. In some embodiments, the lateral flow assay runs at about 25° C. In some embodiments, the lateral flow assay runs at ambient or room temperature. In some embodiments, the ambient or room temperature is about 25° C.

In some embodiments, the lateral flow assay includes multiple detection antibodies and conjugates to differentiate between different dermatophyte organisms in a subject sample. In some embodiments, cellulose nanobeads are used in the lateral flow assay of the present disclosure, and the use of different colored CNBs allows this differentiation of or detection of different dermatophyte organisms in the subject sample. In some embodiments, the different capture antibodies are striped onto a different portion of the nitrocellulose membrane in the lateral flow assay to produce multiple detection lines. In some embodiments, the different detection antibodies conjugated to differently colored CNBs are flowed over the striped antibody (ies) on the nitrocellulose membrane to produce a different color when they each bind at one detection line (e.g. a blue NCB and a yellow NCB on the same line appears green).

### Organisms

In some embodiments, dermatophyte organisms detected using the compositions and methods of the present disclosure include, but are not limited to, Trichophyton fungi, Microsporum fungi, Epidermophyton fungi, Microsporum amazonicum, Microsporum audouinii, Microsporum boullardii, Microsporum canis, Microsporum canis var. distortum, Microsporum cookei, Microsporum distortum, Microsporum duboisii, Microsporum equinum, Microsporum ferrugineum, Microsporum fulvum, Microsporum gallinae, Microsporum gypseum, Microsporum langeronii, Microsporum nanum, Microsporum persicolor, Microsporum praecox, Microsporum ripariae, Microsporum rivalieri, Epidermophyton floccosum, Epidermophyton stockdaleae, Trichophyton benhamiae, Trichophyton bullosum, Trichophyton concentricum, Trichophyton equinum, Trichophyton eriotrephon, Trichophyton erinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, andTrichophyton violaceum. In some embodiments, the dermatophyte organism is Trichophyton benhamiae, Trichophyton bullosum, Trichophyton concentricum, Trichophyton equinum, Trichophyton eriotrephon, Trichophyton erinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, or Trichophyton violaceum. In some embodiments, the dermatophyte organism is T. rubrum, T. interdigitale, E. floccosum, or M. canis.

### Infections

In some embodiments, infections detected using the compositions and methods of the disclosure include an infection caused by one or more dermatophyte organisms including, but not limited to, Trichophyton fungi, Microsporum fungi, Epidermophyton fungi, Microsporum amazonicum, Microsporum audouinii, Microsporum boullardii, Microsporum canis, Microsporum canis var. distortum, Microsporum cookei, Microsporum distortum, Microsporum duboisii, Microsporum equinum, Microsporum ferrugineum, Microsporum fulvum, Microsporum gallinae, Microsporum gypseum, Microsporum langeronii, Microsporum nanum, Microsporum persicolor, Microsporum praecox, Microsporum ripariae, Microsporum rivalieri, Epidermophyton floccosum, Epidermophyton stockdaleae, Trichophyton benhamiae, Trichophyton bullosum, Trichophyton concentricum, Trichophyton equinum, Trichophyton eriotrephon, Trichophyton erinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, and Trichophyton violaceum. In some embodiments, the dermatophyte organism is Trichophyton benhamiae, Trichophyton bullosum, Trichophyton concentricum, Trichophyton equinum, Trichophyton eriotrephon, Trichophyton erinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, or Trichophyton violaceum. In some embodiments, the dermatophyte organism is T. rubrum, T. interdigitale, E. floccosum, or M. canis.

In some embodiments, the infections detected using the compositions and methods of the disclosure include, but are not limited to, tinea pedis (athlete's foot), tinea corporis (ringworm of the body), tinea cruris (jock itch), tinea capitis (blackdot ringworm), dermatophyte onychomycosis (ringworm of the nail), tinea unguium, Majocchi's granuloma, infectious folliculitis, tinea facici (facial ringworm), tinea manuum (ringworm of the hands), tinea barbae, and tinca incognito. In some embodiments, the subject is suspected of having an infection based on symptoms. In some embodiments, the subject is suspected of having an infection based on known exposure to a causative dermatophyte. In some embodiments, the subject is suspected of having an infection based on suspected exposure to a causative dermatophyte.

In some embodiments, the compositions and methods disclosed herein are used to diagnose a dermatophyte infection in any appropriate subject. In some embodiments, the subject is a mammal. In some embodiments, the subject is human. In some embodiments, the subject is zoonotic or an agricultural mammal. In some embodiments, the subject is canine, feline, equine, bovine, or porcine.

In some embodiments, the subject has already been diagnosed with a different fungal infection. In some embodiments, the subject is already receiving treatment with an antifungal agent. In some embodiments, the anti-fungal agent is systemic. In some embodiments, the antifungal agent is local or topical. In some embodiments, the compositions and methods disclosed herein are used to confirm a diagnosis of a subject having a dermatophyte infection. In some embodiments, after initial diagnosis, the compositions and methods of the present disclosure are used to monitor treatment of a dermatophyte infection.

“Antifungal agent” means an agent that inhibits growth of or kills a fungus. Types of antifungal agents that are useful in the present disclosure include, but are not limited to, tavaborole, nystatin, candicidin, amphotericin B, filipin, bifonazole, albaconazole, and abafungin. Other examples of antifungal agents can be found in Dixon and Walsh, Medical Microbiology 4th edition, Chapter 76 “Antifungal Agents).

### Kits

In yet another aspect, the disclosure includes a kit for determining whether a sample contains a dermatophyte organism. In some embodiments, the kit comprises the reagents and antibodies for immunohistochemistry analysis and instructional materials for the use thereof. In some embodiments, the kit comprises a lateral flow assay cassette of the instant disclosure; and instructional materials for the use thereof. In some embodiments, the kit comprises a lateral flow assay cassette of the instant disclosure, a sample buffer, and instructional materials for the use thereof.

### EXAMPLES

### Example 1-Immunohistochemical Diagnosis of Onychomycosis by Monoclonal Antibodies Detection of Dermatophyte T. rubrum

Onychomycosis is the most common nail infection caused by multiple strains of fungi, predominantly by dermatophyte Trichophyton rubrum. Currently, Periodic Acid-Schiff (PAS) staining is the gold standard for histological onychomycosis detection. However, it does not differentiate between the types of fungal species invading the nail plate and bed (Tchernev 2013) A new immunohistochemical (IHC) method can help pathologists differentiate between the various fungal strains. Using monoclonal antibodies, this experiment aimed to differentially detect T. rubrum via a new type of staining protocol. By discerning the causative species, physicians can provide a targeted and effective therapy for patients. Moreover, the lateral flow assays developed using the present monoclonal antibodies and methods may be used to differentiate between different dermatophyte infections. For example, Table 1 demonstrates several monoclonal antibodies that bind to particular organisms. This allows a multiplex assay to be performed where multiple dermatophyte organisms may be detected in a single lateral flow assay. Additionally, given the specific binding of monoclonal antibodies, these lateral flow assays may be used to differentiate dermatophyte infections from other fungal infections (e.g. Candida infections) to prevent patients from being treated with inappropriate therapies that will not be effective against infection.

This experiment demonstrates the isolation and validation of various mice monoclonal antibody (mAb) candidates for T. rubrum.

Isotype 17B6 displayed the highest binding pair signal to the analyte. Direct ELISA of mAb 17B6 demonstrates immunoreactivity to T. rubrum as well as other lesser common fungal species.

### Objective

To develop a monoclonal antibody stain that can be used to drastically improve histological findings of T. rubrum, the most common species found in onychomycosis in a clinical setting.

### Methods

Histology slides that were analyzed for routine onychomycosis analysis were subjected to secondary analysis. All samples were previously confirmed for onychomycosis by PAS stain and molecular PCR. 148 retrospective, and de-identified, unstained toenail histology slides from 37 previous patients were stained. 21 patients were known to have tested positive for onychomycosis caused by T. rubrum, 5 by C. albicans (yeast), 5 by T. interdigitale (dermatophyte), and 6 negative controls. Monoclonal antibody stains were optimized to Quantum HDx.

### IHC Assay

1. Use paraffin-embedded sections. Section 3-4 microns thick ribbons onto slide.
2. Bake slides horizontally in the oven at 70° C. for 1 hour.
3. Reagents placed in Quantum HDx. 1:100 dilution for antibody stains with TBS buffer.
4. Incubate section with DS1 buffer for 5 min. TBS buffer wash 3 times.
5. Incubate slides with T. rubrum antibody at 25° C. for 45 min. Wash 1 time.
6. Incubate for 12 min with Polymer Enhancer.
7. Incubate for 20 min with APx-Polymer-2. Wash 2 times.
8. Incubate for 12 min with Red Substrate.
9. Incubate for 12 min with Red Chromogen.
10. Counterstain with Hematoxylin. Wash 1 time.
11. Dip finished slides in water. Leave dry and then dip in xylene.
12. Mount with Acrymount Mounting Solution

Histological findings indicate the IHC monoclonal antibody staining technique was found to be more discriminatory than original PAS slides when used to identify dermatophyte structures.

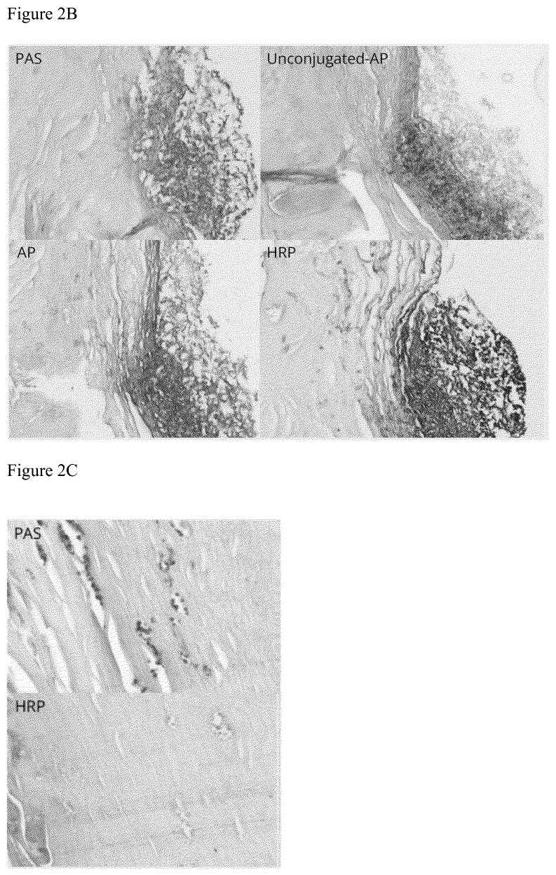

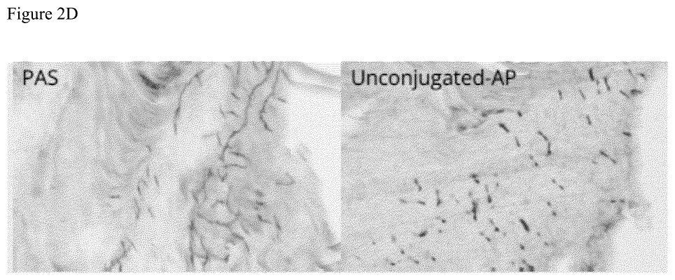

Typically, the PAS stain relies on the presence of polysaccharide structures of the cell wall to stain fungal structures. The antibody stain exhibited high binding specificity to dermatophyte cell wall, allowing it to produce strong chromogenic development and, thus, gave it the capability to identify nuanced fungal elements that do not exhibit conventional fungal cell structure. Fungal elements appear stained using the IHC technique, while these same fungal elements did not appear stained using the PAS technique (FIG. 2B). This demonstrates that these unique structures are degraded fungal elements which may be the result of destroyed fungal structures, and lack polysaccharide elements in their cell wall.

### Quantitative Interpretation

The data determined that this monoclonal antibody stain could not distinguish between T. rubrum and T. interdigitale (p=0.823). However, other antibodies disclosed herein may be able to distinguish between different dermatophyte organisms. Further, significant findings were found when differentiating between T. rubrum and C. albicans (p=0.026) and negatives (p=0.007). When comparing dermatophytes to negative controls and yeast, our stain had a strong statistical significance (p=0.003). Dermatophyte sensitivity is 98.7%, specificity=74.0%, PPV=85.7%, and NPV=97.4%. The results indicate that, using monoclonal antibodies, dermatophyte structures are able to be differentially detected.

TABLE 1
McNemar's Statistical Analysis

| | Sensitivity | Specificity | PPV | NPV | |
|---|---|---|---|---|---|
| Test | (%) | (%) | (%) | (%) | P-value |
| T. rubrum vs. | 100 | 71.9 | 88.2 | 100 | 0.0077 |
| Negative | | | | | |
| T. rubrum vs. | 98.5 | 60.9 | 88.2 | 93.3 | 0.0269 |
| C. albicans | | | | | |
| T. rubrum vs. | 85.9 | 30.8 | 88.2 | 26.7 | 0.8231 |
| T. interdigitale | | | | | |
| Dermatophyte | 98.7 | 74.0 | 85.7 | 97.4 | 0.003 |
| vs. Non- | | | | | |
| dermatophyte | | | | | |

### CONCLUSION

The 17B6 antibody stain was able to selectively stain dermatophyte structures. Based upon this finding, the antibody stain is able to stain dermatophytes, especially T. rubrum of which the cell wall has been destroyed and does not show up on microscopy with the classical branching hyphae structure. In the PAS stain, these potentially degraded fungal structures do not definitively suggest the presence of fungal elements and, thus, cannot be not diagnosed as positive. However, when stained with mAb stain, the potentially degraded fungal structures are stained brightly red, pink, or brown, indicating the presence of dermatophyte structures. This gives the 176B monoclonal antibody stain an edge over the PAS stain.

### Example 2-Identification of Dermatophyte Using Gold Conjugate in the Lateral Flow Assay

To verify that a gold-based assay conditions could accurately identify positive and negative fungal nail samples, a lateral flow assay using mAb 17B6 as disclosed herein was performed.

An Ahlstrom 6614 membrane was sprayed with 8 μl/cm OD 10. 417B6 gold conjugate, and a CN95 nitrocellulose membrane was striped with 17B6 at a concentration of 1 mg/mL and GAM IgG control antibody at a concentration of 2 mg/mL.

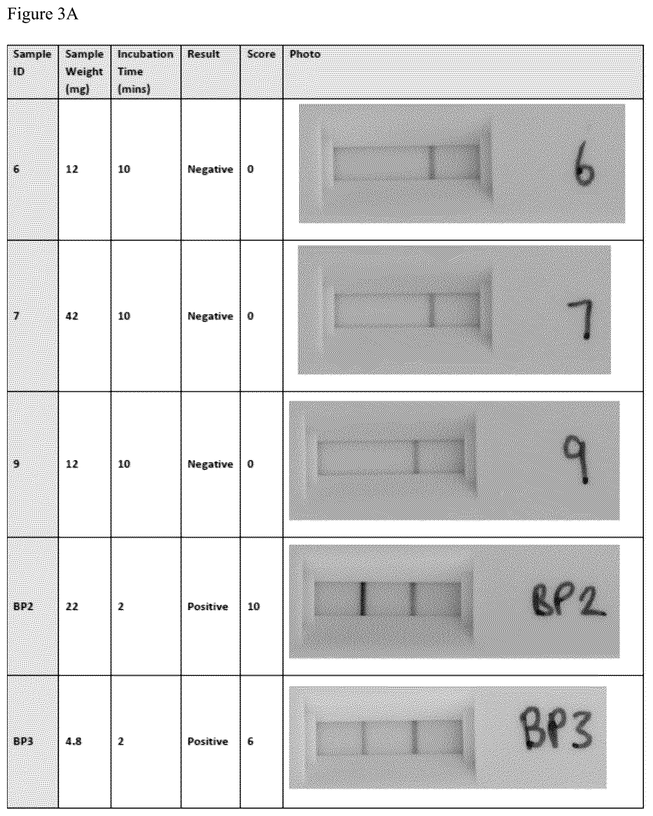

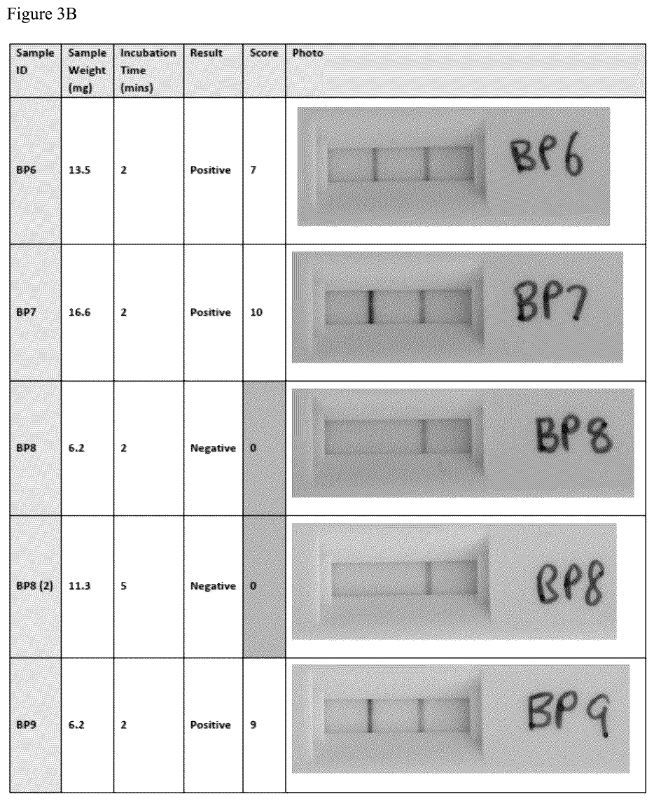

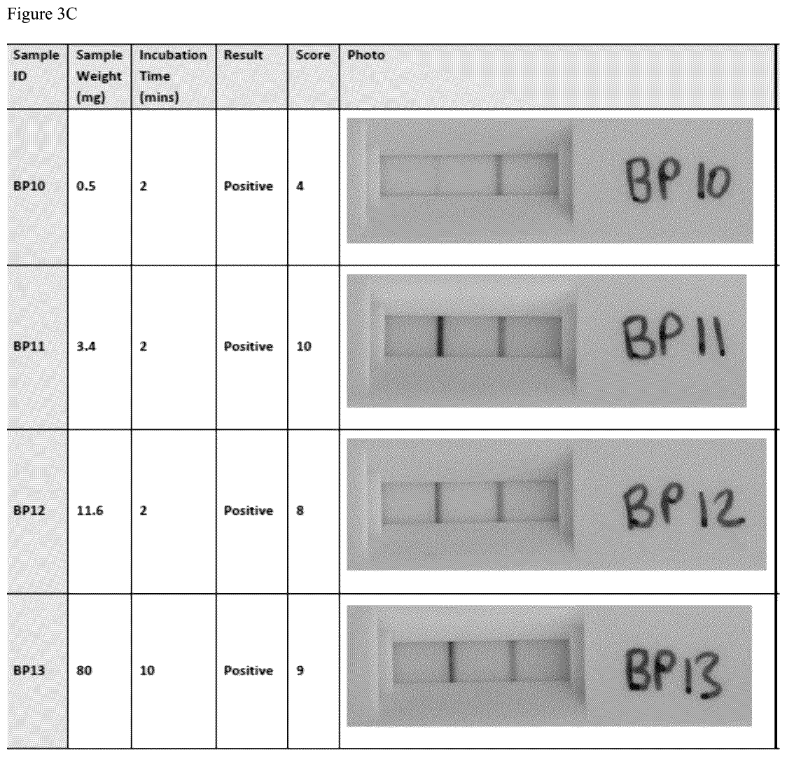

Card Assembly: The nitrocellulose membrane was adhered onto the center portion of a backing card, with the sample pad and absorption pad adhered on either side of the nitrocellulose membrane (with an overlap of the membrane by +2 mm) and the wick pad placed on the opposite end of the sample pad (see for example, FIG. 1). The cards were cut into 1.93 mm strips using a Biodot cutter, and the strips were placed into MICA 200 cassettes and sealed using a cassette scaler. The cassettes were placed in a foil bag with desiccant for later use. Sample preparation: Known positive and negative infected nail samples were obtained. Samples 6, 7, and 9 are known to be negative. All other samples were positive for T. rubrum infection. A piece of nail sample was weighed and placed into a 2 mL Eppendorf tube. 250μ L of sample buffer (2% Tergitol buffer in 1×PBS) was added to each tub, and allowed to soak for the time specified in FIG. 3A-C (Incubation Time). 100 μl was removed and added to the test card and allowed to run for 10 minutes before being scored and photographed.

Conclusion: The gold-based assay correctly identified all three negative samples (see FIG. 3A-C). Of the 10 positive samples, 9 were correctly identified with only sample BP8 coming up as negative. Using the fungal extract, the assay detection limit was determined to be in the range of 30-60 ng/mL. The BP8 sample was re-run with 2.5× longer incubation time, but still came up negative. This sample was later shown to be negative by PCR (data not shown), and appears to have been mislabeled as a positive sample before this experiment was performed. This shows the power of the lateral flow assay which, correctly, identified this sample as negative. The lateral flow assay properly identified all negative and positive samples tested.

### Example 3-Optimization of mAb Passive Gold Conjugates

The purpose of this experiment was to demonstrate the optimal pH and antibody ratio for passive conjugation of monoclonal antibodies of the disclosure to colloidal gold. Method:

mAb: protein A purified monoclonal (mouse) antibody from cell line 17B6.1E3 stored at −20° C. (1.4 mg/mL); protein A purified monoclonal (mouse) antibody from cell line 8B10.1G6.1B5.1G3.D6 stored at −20° C. (2.68 mg/mL); IgM purified monoclonal (mouse) antibody from cell line 13E10.1E2.1D4.1F3.E2 stored at −20° C. (3.83 mg/mL).

Colloidal gold: DCN colloidal gold (Au), OD 1.11 at 523 nm, P/N CG-10.

Buffers: 100 mM sodium phosphate buffer, pH 6.0; 100 mM sodium phosphate buffer, pH 7.0; 100 mM; Borate buffer, pH 8.0; 100 mM Borate Buffer pH 9.0; 10% sodium chloride (1.7M NaCl).

300 μl of 100 mM sodium phosphate buffer, pH 6.0 was added to 3 mL of the OD1 Gold solution and mixed. This step was repeated using the 100 mM sodium phosphate buffer pH 7, pH 8, and pH 9, and the borate buffers. This adjusted the gold solution to the appropriate conjugation pH. 100 μl of antibody solution was prepared to 100 μg/mL in each borate buffer (pH 6, pH 7, pH 8, and pH 9). The antibody solutions were added to a microwell plate per the table below:

| Conjugation | Ab load per mL of OD1 Gold | | | | | | | | |
|---|---|---|---|---|---|---|---|---|---|
| pH | 0 μg/mL | 1 μg/mL | 2 μg/mL | 4 μg/mL | 6 μg/mL | 8 μg/mL | 10 μg/mL | 12 μg/mL | |
| Phosphate pH | 0 μL | 2 μL | 4 μL | 8 μL | 12 μL | 16 μL | 20 μL | 24 μL | Volume |
| 6 | | | | | | | | | of |
| Phosphate pH | 0 μL | 2 μL | 4 μL | 8 μL | 12 μL | 16 μL | 20 μL | 24 μL | 100 μg/ |
| 7 | | | | | | | | | mL Ab |
| Borate pH 8 | 0 μL | 2 μL | 4 μL | 8 μL | 12 μL | 16 μL | 20 μL | 24 μL | solution |
| Borate pH 9 | 0 μL | 2 μL | 4 μL | 8 μL | 12 μL | 16 μL | 20 μL | 24 μL | to add |

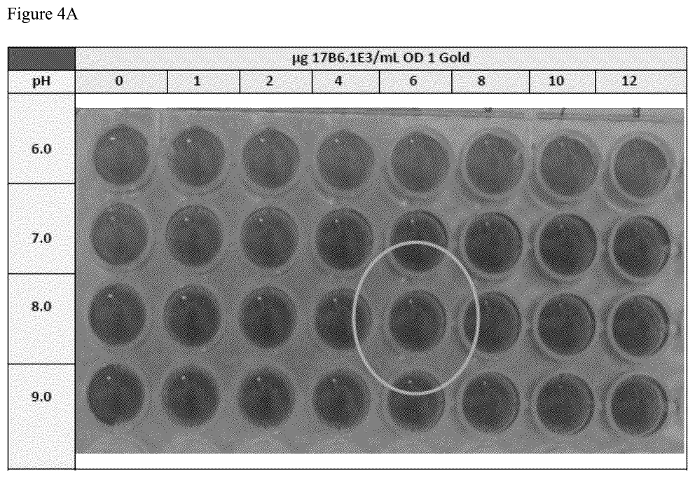

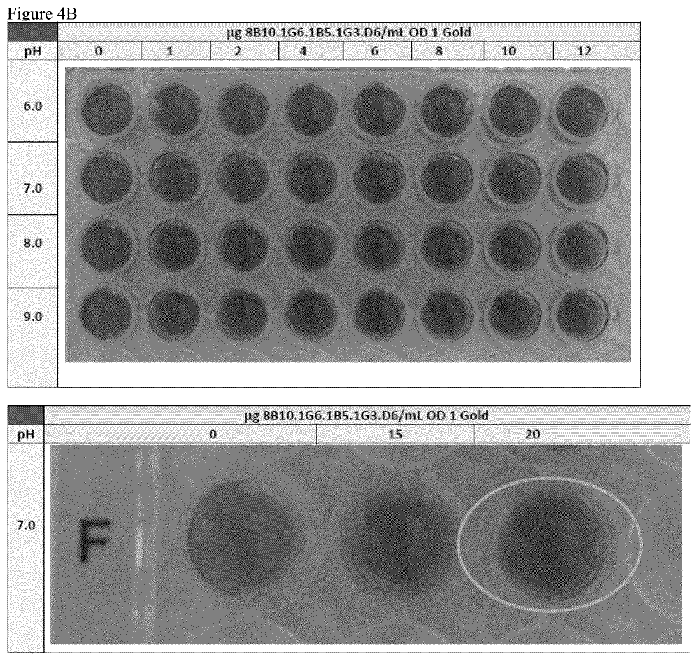

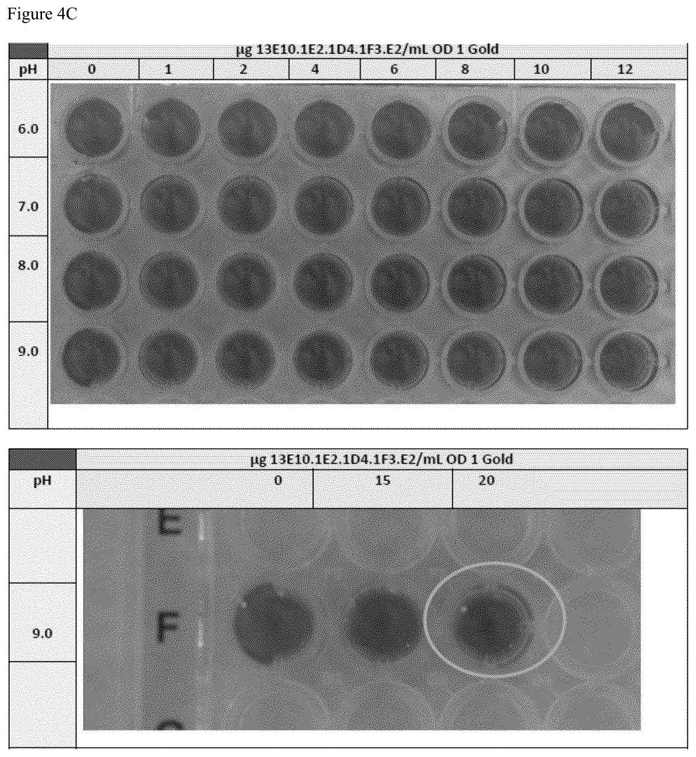

200 μl of OD1 gold at pH 6 (see above) was added to each well in the first row and mixed. This was repeated with each pH level of gold. Once the gold was added to the antibody solution and mixed, the mixture was incubated for 5 minutes at room temperature. After incubation, 20 μl of 10% NaCl solution was added to each well and mixed. This mixture was incubated for 5 minutes at room temperature, and the plate was then read by observing the colors of each well. See FIG. 4A-D. The wells that show no color change provide a stable conjugate, while those wells that change to purple, blue, or grey in the presence of NaCl provide evidence that the antibody load and/or conjugation pH used were unable to produce enough coverage to protect the gold from salt-induced aggregation.

### Example 4-Assay Development for Detection of Trichophyton rubrum

This experiment tested the sensitivity of 11 mouse monoclonal antibodies against Trichophyton rubrum to develop a custom sandwich ELISA and lateral flow assay (LFA). Each of the mouse monoclonal antibodies were purified and biotin conjugated.

TABLE 2
anti- T. rubrum monoclonal antibodies

| [symbol] lone ID | | [symbol] nconj. Ab | [symbol] iotinylated | [symbol] mount of Ab |
|---|---|---|---|---|
| Abbreviation | [symbol] bbreviation | Conc. Mg/ml | Ab Conc. Mg/ml | biotinylated (mg) |
| [symbol] 4F5.1E3.2H2 | [symbol] 4F5 | [symbol] .8 | [symbol] .83 | |
| [symbol] 0B12.1C4.1C3 | [symbol] 0B12 | [symbol] .13 | [symbol] .94 | |
| [symbol] B10.1G6.1B5.1G3.D6 | [symbol] B10 | [symbol] .68 | [symbol] .41 | [symbol] .5 |
| [symbol] 0G6.F3.2E11 | [symbol] 0G6 | [symbol] .07 | [symbol] .9 | |
| [symbol] 9F6.G4.2B4 | [symbol] 9F6 | [symbol] .65 | [symbol] .41 | [symbol] .5 |
| [symbol] B9.G12.2C7 | [symbol] B9 | [symbol] .33 | [symbol] .41 | [symbol] .5 |
| [symbol] 7E8.2E2.1B2 | [symbol] 7E8 | [symbol] .7 | [symbol] .41 | [symbol] .5 |
| [symbol] 9D9.1G4.2D6 | [symbol] 9D9 | [symbol] .7 | [symbol] .41 | [symbol] .5 |
| [symbol] 7B6.1E3 | [symbol] 7B6 | [symbol] .04 | [symbol] .87 | |
| [symbol] 3E10.1E2.1D4.1F3.E2 | [symbol] 3E10 | [symbol] .27 | [symbol] .83 | |
| (IgM) | | | | |
| [symbol] 6G3.2G9.B2.1E5.1B5 | [symbol] 6G3 | [symbol] .83 | [symbol] .83 | |
| (IgM) | | | | |

*all antibodies are IgG1/kappa unless otherwise indicated [symbol]
indicates data missing or illegible when filed

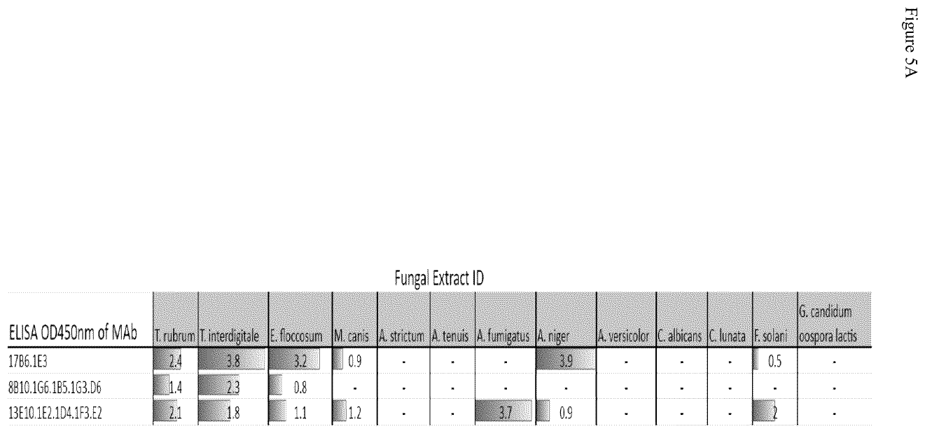

FIG. 5A and FIG. 5B show the binding of the different antibodies to dermatophytes and unrelated organisms.

Antibodies 24F5 and 27E8 were removed from further consideration for use in the lateral flow assay due to non-specific interaction of paired antibodies in the absence of analyte (data not shown).

TABLE 4

| | | | [symbol] aired | | |
|---|---|---|---|---|---|
| | | [symbol] aired | with | | |
| [symbol] apture | | OD450 | Detection | | |
| Ab ID | [symbol] sotype | nm | Ab ID | [symbol] sotype | [symbol] ote |
| [symbol] 7B6 | [symbol] gG1/kappa | [symbol] .35 | [symbol] 7B6 | [symbol] gG1/kappa | [symbol] elf pair |
| [symbol] 3E10 | [symbol] gM | [symbol] .52 | [symbol] 3E10 | [symbol] gM | [symbol] elf pair |
| [symbol] 7B6 | [symbol] gG1/kappa | [symbol] .6 | [symbol] 3E10 | [symbol] gM | [symbol] gG-IgM |
| [symbol] 3E10 | [symbol] gM | [symbol] .12 | [symbol] 7B6 | [symbol] gG1/kappa | [symbol] gM-IgG |
| [symbol] B10 | [symbol] gG1/kappa | [symbol] .69 | [symbol] 7B6 | [symbol] gG1/kappa | [symbol] gG-IgG |

*bolded values—high binding pair signal > 1.5 [symbol]
indicates data missing or illegible when filed

Based on the results here (Table 4), antibodies 17B6, 13E10, and 8B10 were selected for further testing.

### Example 5-Testing Buffers for Detection of T. rubrum in Patient Samples

A checkerboard sandwich ELISA was run using 11 unconjugated and 11 biotin-conjugated mouse monoclonal antibodies against T. rubrum which resulted in the selection of 5 best pairs of capture detection antibodies for the specific detection of T. rubrum. This ELISA was then used for T. rubrum in patient samples. Before this testing, an assay was run to assess the effect of different buffers that might be used for patient sample preparation/extraction. Here, the matrix effect of 4 different buffers on the detection of T. rubrum was explored in sandwich ELISA.

Materials: Coating Buffer-NaHCO₃buffer, pH 9.2
Blocking Buffer-3% Fish gel in 1×PBS
SA-HRP
Assay Buffer-1% Fish gel in 1×PBS
0.5% tergitol in PBS
1.5% lauryl maltoside in PBS
1% digitonin in PBS
1.5% CHAPS in PBS

### Procedure:

Unconjugated 17B6, 13E10, and 8B10 were coated at 5 μg/ml (100 μl/well) in coating buffer pH 9.2 overnight. The plate was washed 4 times with 1×PBST, and the plate was blocked with 3% Fish Gel in 1×PBS at 300 μl/well for 1 hour shaking at 37° C. The plate was washed again in 1×PBST, and 50 ng/well (100 μl) of T. rubrum antigen in 1% Fish gel in 1×PBS buffer, 0.5% tergitol, 1.5% lauryl maltoside, 1% digitonin, or 1.5% CHAPS was added as shown in Table 5. These mixtures were incubated for 1 hour at room temperature with shaking. As shown in Table 5, columns 1-5 are neat (e.g. undiluted) buffers and columns 6-10 are diluted 1:10. The plate is washed again in 1×PBST, and biotin-labelled 17B6 or 13E10 were added at 1 μg/ml (100 μl/well) in 1% Fish gel in 1×PBS, 0.5% tergitol buffer, 1.5% lauryl maltoside, 1% digitonin, or 1.5% CHAPS as shown in Table 5 for 1 hour at room temperature with shaking. The plates were washed with 1×PBST, and SA-HRP was added at 1:50,000 (100 μl/well) in 1% Fish gel in 1×PBS, 0.5% tergitol buffer, 1.5% lauryl maltoside, 1% digitonin, or 1.5% CHAPS for one hour at room temperature with shaking. The plate is washed in 1×PBST and the substrate TMB is added at 100 μl/well for 30 minutes at room temperature in the dark. The reaction was stopped with IN HCL and the plates were read at 450 nm.

TABLE 5

| | | Neat Buffer | | | | 1:10 (buffers:Rkl) | | | |
|---|---|---|---|---|---|---|---|---|---|
| | A450 nm | | Lauryl | | | | Lauryl | | |
| Capture/Detection | Rkl | Tergitol | maltoside | Digitonin | CHAPS | Tergitol | maltoside | Digitonin | CHAPS |
| 17B6/17B6 | 3.39 | 3.35 | 3.36 | 3.33 | 3.07 | 3.30 | 3.37 | 3.36 | 3.32 |
| 13 E 10/13E 10 | 0.90 | 3.14 | 0.55 | 1.20 | 0.56 | 2.94 | 0.49 | 0.79 | 0.97 |
| 17B6/13E 10 | 1.57 | 3.27 | 1.12 | 0.96 | 0.76 | 2.97 | 1.20 | 1.19 | 1.52 |
| 13E 10/17B6 | 2.53 | 3.33 | 2.19 | 2.53 | 1.79 | 2.62 | 2.06 | 2.48 | 2.55 |
| 8B10/17B6 | 0.58 | 1.19 | 0.53 | 0.46 | 0.17 | 0.90 | 0.59 | 0.44 | 0.52 |

Results: Of the 4 buffers tested, 1% digitonin in PBS produced the most similar signal strength readings when compared to 1% Fish gel in PBS. The second-best buffer was 1.5% CHAPS, except for the 8B10/17B6 pair which resulted in readings below background when used undiluted, but not when diluted 10 times. 1.5% lauryl maltoside also produced similar results as 1% Fish Gel in PBS (“Rkl Buffer”) except for the 13E10/13E10 pair which demonstrated OD values of 0.5 compared to 1% Fish gel in PBS. While 0.5% tergitol produced very high readings, the solution was cloudy, and the readings were greater than those observed using standard buffer—these readings could be an artefact. This matrix testing was refined to further elucidate the best buffer to use in the lateral flow assays.

Since digitonin is toxic and difficult to handle, it was excluded from the further matrix testing. This assay also included a no antigen control to verify the reactions observed are specific to T. rubrum in the presence of the buffers tested.

The sandwich ELISA was performed with 6 pairs of capture and detection antibodies and the effect of three different kinds of extraction buffer were compared to 1% Fish gel in 1×PBS.

Materials: Coating Buffer-NaHCO₃buffer, pH 9.2
Blocking Buffer-3% Fish gel in 1×PBS
SA-HRP
Assay Buffer-1% Fish gel in 1×PBS
0.5% tergitol in PBS
1.5% lauryl maltoside in PBS
1.5% CHAPS in PBS

The assay was set up as follows. Rows 1 & 2 contained 0.5% tergitol (1:10 tergitol: Rkl); Rows 3&4 contained 1.5% lauryl maltoside (1:10 lauryl maltoside: rkl); Rows 5&6 contained 1.5% CHAPS (1:10, CHAPS: Rkl), and Rows 7&8 contained 1% Fish gel in PBS. The capture Ab-biotinylated Ab=500 ng/well (5 μg/ml); T. rubrum antigen was used at 50 ng/well (500 ng/ml); and the detection Ab-biotinylated Ab was used at 100 ng/well (1 μg/ml).

Capture/Detection pairs: 17B6/17B6; 13E10/13E10; 17B6/13E10; 13E10/17B6; 8B10/17B6; and 8B10/8B10.

Columns 1-6 received T. rubrum antigen while columns 7-12 had no antigen.

The method was performed as described above, and the results shown in Table 6

### Results

TABLE 6
Ab pairs (Capture/Detection)

| | 17B6/ | 13 E 10/ | 17B6/ | 13E 10/ | 8B10/ | 8B10/ | 17B6/ | 13 E 10/ | 17B6/ | 13E 10/ | 8B10/ | 8B10/ |
|---|---|---|---|---|---|---|---|---|---|---|---|---|
| Buffers | 17B6 | 13E 10 | 13E 10 | 17B6 | 17B6 | 8B10 | 17B6 | 13E 10 | 13E 10 | 17B6 | 17B6 | 8B10 |
| Tergitol (final | 3.09 | 2.49 | 2.90 | 2.04 | 2.98 | 1.65 | 0.21 | 2.03 | 2.13 | 0.30 | 0.28 | 0.40 |
| 0.05%) | 3.20 | 2.53 | 2.94 | 2.16 | 2.90 | 1.74 | 0.21 | 2.09 | 2.22 | 0.22 | 0.24 | 0.36 |
| Lauryl Maltoside | 3.33 | 1.13 | 1.77 | 2.48 | 3.15 | 1.78 | 0.07 | 0.06 | 0.06 | 0.07 | 0.09 | 0.11 |
| (final 0.15%) | 3.34 | 1.12 | 1.79 | 2.45 | 3.13 | 1.74 | 0.08 | 0.06 | 0.06 | 0.07 | 0.08 | 0.10 |
| CHAPS (final | 3.17 | 0.93 | 1.17 | 1.89 | 2.69 | 1.27 | 0.07 | 0.06 | 0.05 | 0.06 | 0.07 | 0.07 |
| 0.15%) | 3.15 | 0.92 | 1.16 | 1.94 | 2.68 | 1.24 | 0.07 | 0.06 | 0.06 | 0.06 | 0.07 | 0.07 |
| Rkl Buffer | 3.26 | 0.94 | 1.16 | 2.19 | 2.50 | 1.47 | 0.07 | 0.06 | 0.06 | 0.06 | 0.08 | 0.09 |
| | 3.21 | 1.00 | 1.22 | 2.14 | 2.54 | 1.50 | 0.08 | 0.07 | 0.06 | 0.07 | 0.08 | 0.11 |

| | |
|---|---|
| With Antigen | Without Antigen |

Of the three buffers tested, 0.05% tergitol in PBS produced non-specific binding, especially with 13E10/13E10 and 17B6/13E10 antibody pairs which resulted in OD readings>2. With the other antibody pairs, the background was observed with OD>0.2. The 0.15% lauryl maltoside and 0.15% CHAPS produced very similar readings when compared with 1% Fish gel in PBS. No background or non-specific binding issues were observed with either 0.15% lauryl maltoside or 0.15% CHAPS buffers. The 8B 10 self pair also worked very well with both 0.15% lauryl maltoside and 0.15% CHPAS buffers.

This assay was further refined to titrate the concentration of T. rubrum antigen to determine the sensitivity of 3 pairs of capture ad detection antibodies.

Materials: Coating Buffer-NaHCO₃buffer, pH 9.2
Blocking Buffer-3% Fish gel in 1×PBS
SA-HRP
Assay Buffer-1% Fish gel in 1×PBS
1.5% lauryl maltoside in PBS
Mouse Ig G whole molecule Rkl-5 mg/ml.

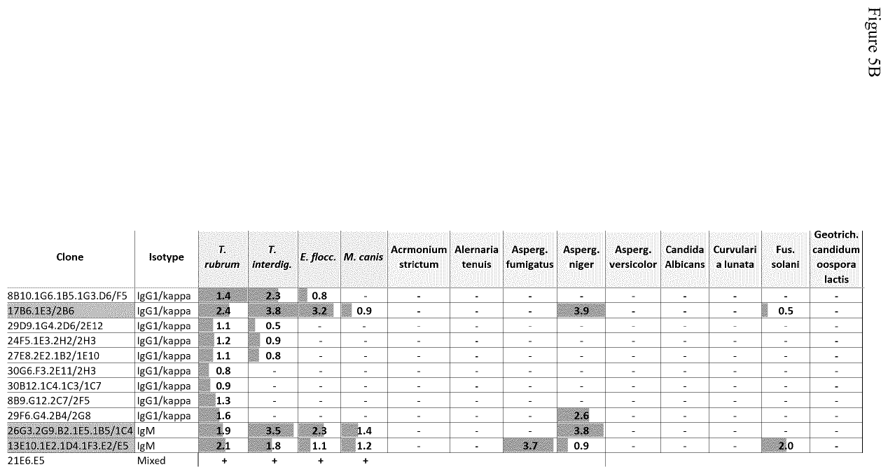

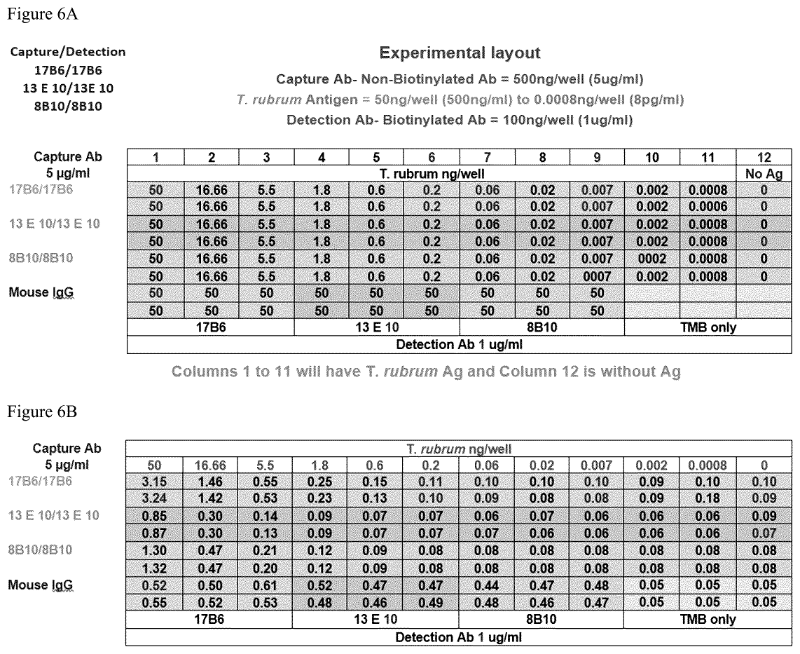

The Experimental layout and results are shown in FIG. 6A-B. Of the three antibody pairs tested, the 17B6 self pair produced better sensitivity down to 50 ng/ml (5.5. ng/well) while 13E10 and 8B10 resulted in positive signals down to 160 ng/ml (16.6 ng/well). The criterion for sensitivity cut-off was 3× over the background (i.e., the rows without antigen). The mouse IgG had a signal of 0.5 with all the 3 pairs at 50 ng/well of T. rubrum tested.

### Example 6-Pilot Testing to Confirm T. rubrum Presence in Patient Samples

This study tested the chosen 17B6/17B6 antibody pair as capture and detection antibodies to confirm the presence of T. rubrum in patient samples extracted in 3 different buffers.

Reagents: patient nail samples: 5 positive for T. rubrum infection and 5 negative for T. rubrum infection. These were processed as 2.4-9 mg nail clippings per tube using 100 μl buffer per 5 mg of sample
Buffer A: 1% Fish Gel in 1×PBS
Buffer B: 0.15% lauryl maltoside in 1% Fish Gel in 1×PBS
Buffer C: 0.15% CHAPS in 1% Fish Gel in 1×PBS

Protein content was determined using all three extraction buffers in all samples (nanodrop ODs were read using 1 Abs=1 mg/mL setting). The positive samples had a higher yield than negative samples Method:

15 tubes were labeled for negative samples (1A, 1B, 1C to 5A, 5B, and 5C) and for positive samples (BPIA, BP1B, BPIC to BP5A, BP5B, BP5C). Each tube was weighed and the weight recorded. Three tubes were prepared for each sample, and each tube was weighed to calculate sample weight. The extraction buffer was added at 100 μL per 5 mg of sample. The volume was adjusted based on weight. The tubes were incubated at room temperature for 30 minutes, with vortexing every 5 minutes for a few seconds. The tubes were spun at 2000 rpm for 10 minutes at 4° C. The supernatant was transferred to a fresh tube and Nanodrop readings were read in duplicate using the setting 1 Abs=1 mg/mL. Protein content was determined using all three extraction buffers in all samples (nanodrop ODs were read using 1 Abs=1 mg/mL setting). The positive samples had a higher yield than negative samples. After determining the protein levels, the samples were pooled as below:
P1=50 μl each of BP1, BP2, BP3, BP4, and BP5 in Buffer A
P2=50 μl each of BP1, BP2, BP3, BP4, and BP5 in Buffer B
P3=50 μl each of BP1, BP2, BP3, BP4, and BP5 in Buffer C
n1=50 μl each of P1, P2, P3, P4, and P5 in Buffer A
n2=50 μl each of P1, P2, P3, P4, and P5 in Buffer B
n3=50 μl each of P1, P2, P3, P4, and P5 in Buffer C
Super pool positive (SP1)=P1, P2 and P3 (200 μl each)
Super pool negative (SN1)=N1, N2, and N3 (200 μl each).

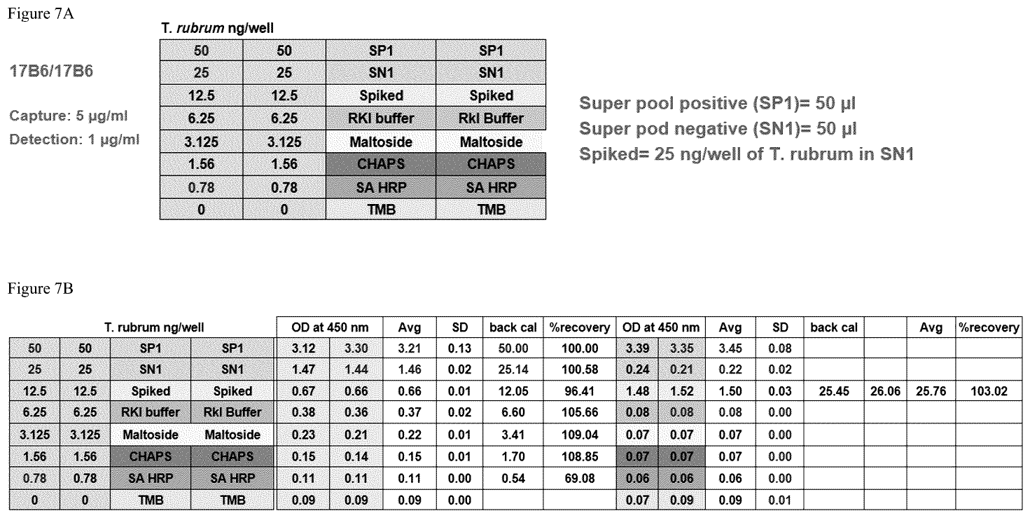

The experimental layout and results are shown in FIG. 7A-B.

Mouse anti-T. rubrum antibody 17B6 was used as capture and detection antibody for these studies. The T. rubrum standard was run in duplicates starting from 50 ng/well to 0.78 ng/well 100 μl/well in 1% fish gel in PBS. The positive and negative super pool samples were added at 100 μl/well in duplicates (50 μl of sample+50 μl of 1% fish gel in PBS). Negative sample SN1 was spiked with 25 ng/well of T. rubrum in duplicate. The 1% fish gel in PBS, maltoside buffer (0.15% in 1% Fish Gel in 1×PBS) and CHAPS buffer (0.15% in 1% Fish Gel in 1×PBS) were tested in duplicate without any antigen as a negative control. SA-HRP and TMP only controls in duplicate were also included.

Results: The positive patient sample super pools had an average OD of 3.45 and the negative samples had an OD of 0.22. The antigen spiked sample in negative patient sample super pools had a recovery of 103%, showing that there was no effect of sample matrix. All three of the buffers tested without antigen also did not show any non-specific reactivity. Overall, the assay clearly identified positive and negative patient samples in a highly specific manner.

This assay was repeated to test the positive and negative samples separately in three different buffers at different dilutions to identify if any of the buffers have any effect on the detection of samples in ELISA.

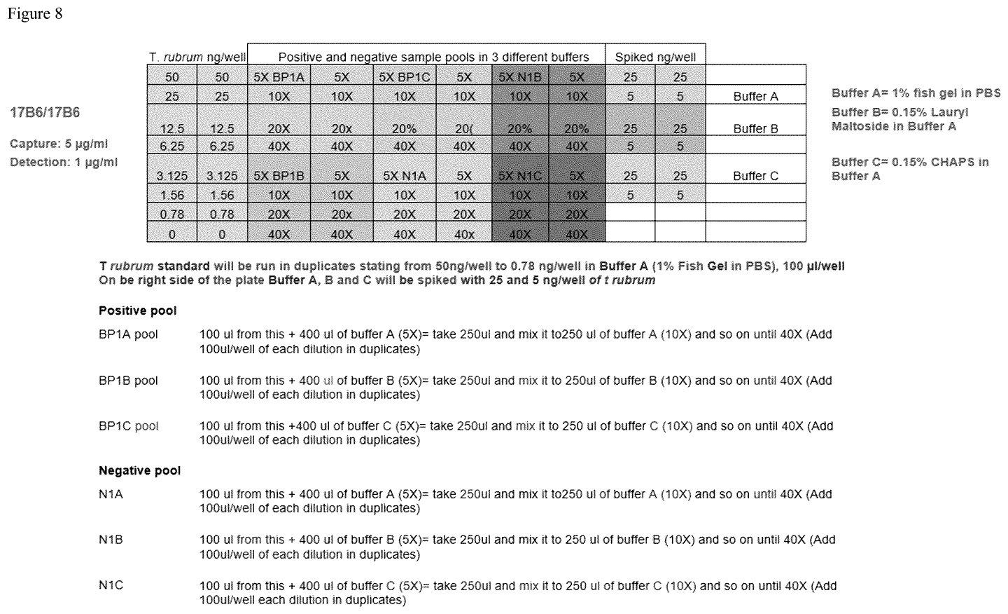

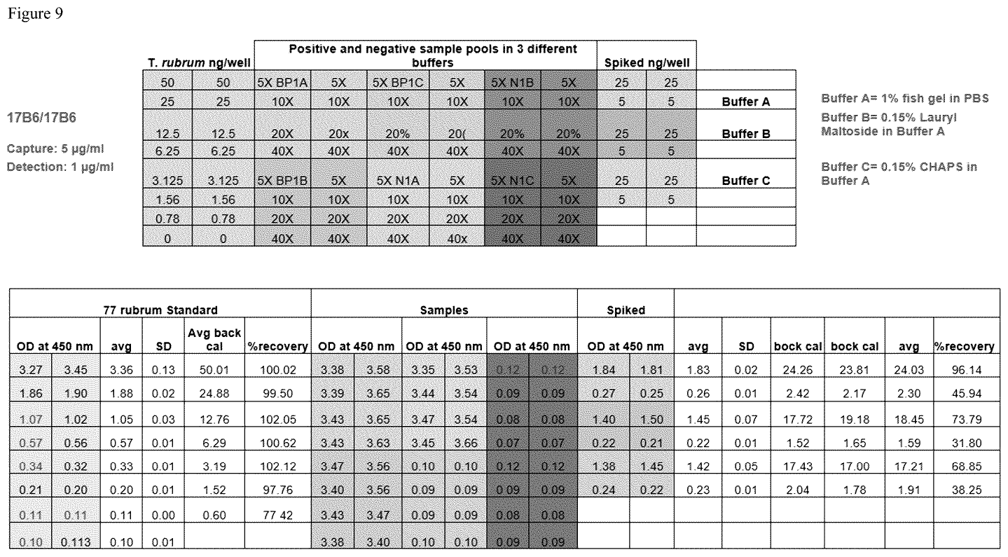

The experimental set up is shown in FIG. 8. The results are shown in FIG. 9.

Results: The positive patient sample pool in all three buffers had an average OD450 nm of 3.49 (maximal signal) in all dilutions tested. The negative sample pool in all three buffers had an average of OD450 nm of 0.1 (background). While sensitivity of the assay could not be determined, these results suggested that extracted patient samples can be highly diluted (>>40×), indicating this assay is very sensitive. Overall, the 17B6/17B6 self pair sandwich ELISA) clearly identified positive and negative patient samples in a highly sensitive manner with all three buffers tested.

### Example 7-Stability Studies

To determine the shelf life of tests at 37° C. and 55° C., tests were performed at predetermined days. Seventeen days at 55° C., and 96 days at 37° C. correlates to around one year's shelf life at ambient temperature.

### Materials and Equipment:

| Description | Lot No. |
|---|---|
| Ahlstrom 6614 sprayed with 8 μL/cm OD 10 17B6 | 1062-067 |
| gold conjugate | |
| CN95 striped 17B6 1 mg/mL and 2 mg/mL GAM IgG | 1062-048 |
| Wick Pad, Ahlstrom 243 (21 mm) | 113517 |
| MIBA Backing Card - MIBA-010 | 076027-01 |
| 2% Tergitol buffer in 1xPBS | 24FEB2021CCB- |
| | 2% |
| Stability Study Controls (1000, 500, 100, 50, 0 ng/ml) | 26MAR2021CCB |
| MICA-200 Cassette, PN MICA-200 TOP/BOTTOM, | LN W19-316 |
| Exp 15OCT2024 | |
| Biodot Guillotine Cutter CM4000 | CM1328 |
| Cassette Press | CP002 |

Method: 100 μl sample is added to the cassette, and after 10 minutes, the tests are scored and photographs are taken.

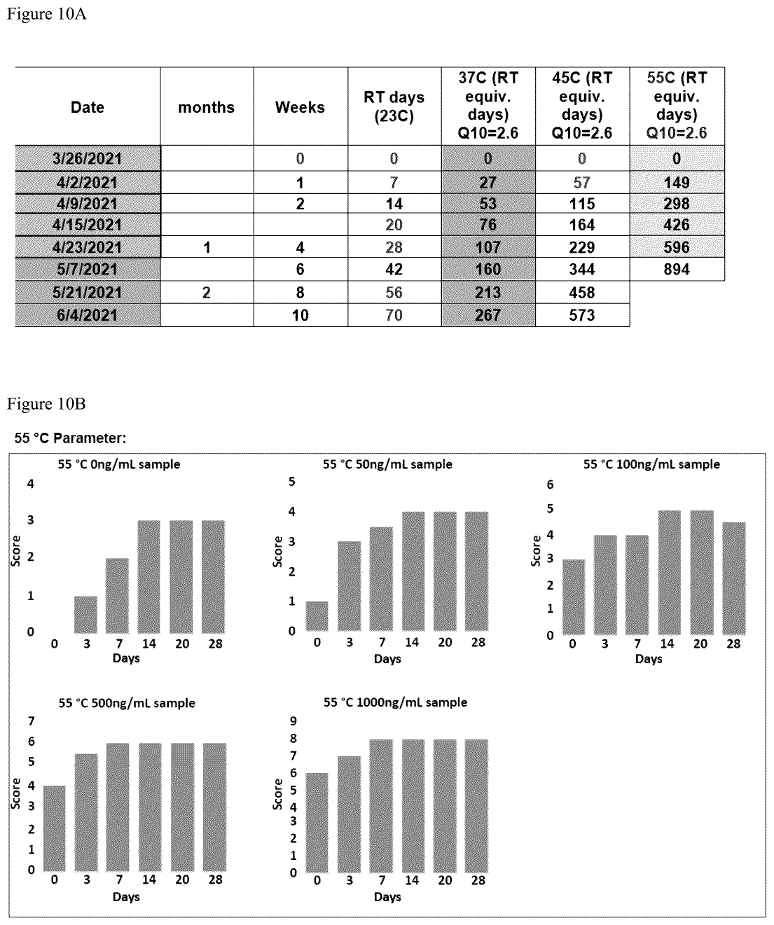

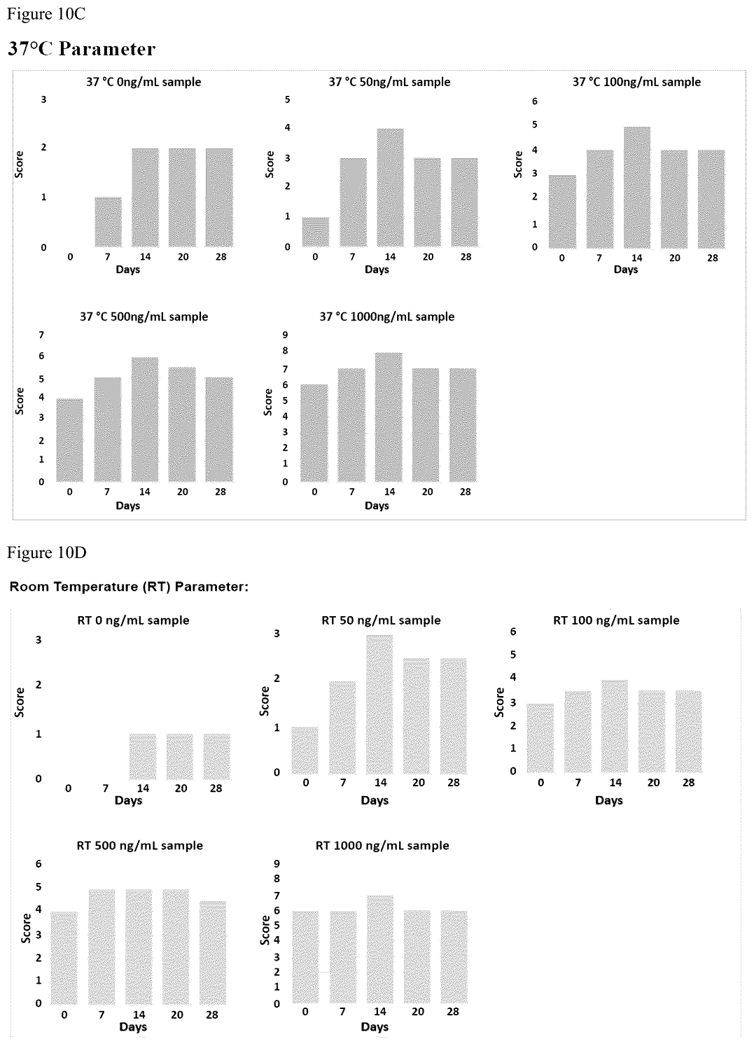

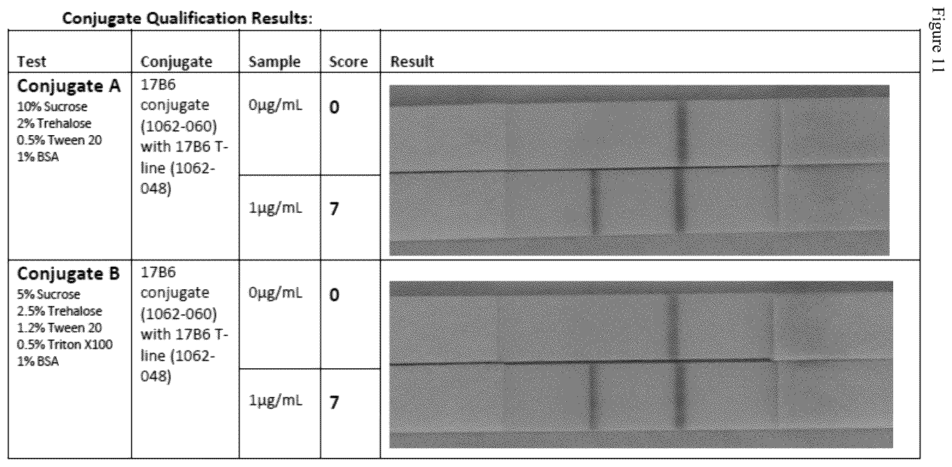

The results are shown in FIG. 10A-D. Sensitivity of the assay increases by 2-3 grades with the tests stressed at 55° C. The results plateaued at day 14, which correlates to +300 days at room temperature. The 55° C. tests reached a score of 3 compared to the room temperature tests at 1, using the negative sample.

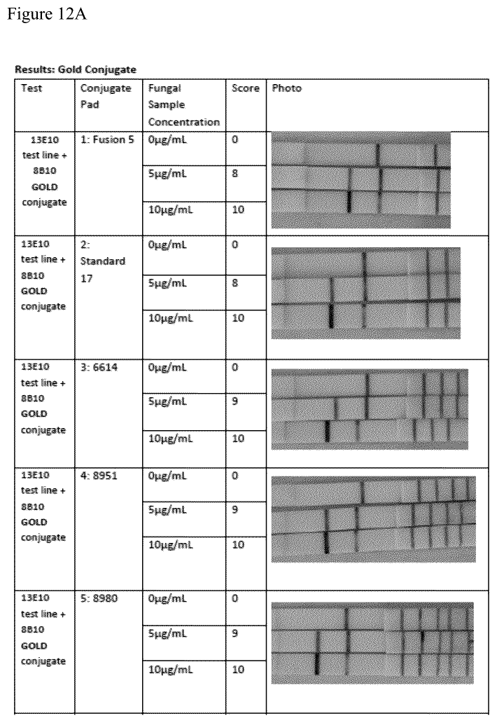

Previous studies (data not shown) demonstrated that test sensitivity increased after 72 hours at 55° C., most likely due to the proteins gaining better adhesion to the membrane when exposed to heat. This can lead to non-specific binding (NSB), which may be observed more with the celllose nanobead (CNB) assay. When the cassettes were opened, it could be seen that the conjugate had aggregated and not released from the conugate pad; this was especially noticble in the tests stressed at 55° C., but was also present in tests stored at room temperature. It was determined that the quantity of sugars present (2.5% sucrose & 1.25% trehalose) was not adequte to protect and help release the conjugate. Similarly, tween 20 was also increased past 0.25%. Following these results, two different conjugate dilution buffers with increased sugars (10% sucrose and 2% trehalose or 5% sucrose and 1.5% trehalose) and surfactants (0.5% Tween 20 and 1.2% Tween 20) were tested. FIG. 11 shows the test buffers and results. The conjugate released completely using both of the conjugate dilution buffers tested, with similar results for both.

The choice of a cellulose nanobead (CNB) or gold as the conjugate was also found to play a role in stability. To test the effect of placing the strip at 55° C. on nonpecific binding a test using 8B10 as the CNB conugate and 17B6 as the test line reagent was used.

### Materials and Equipment:

| Description | Lot No. |
|---|---|
| 8B10 CNB 10:1, 0.03% solids, 8 μL/cm; CN95 | 1062-057 |
| 17B6 1 mg/mL & GAM IgG 2 mg/mL | |
| 8B10 CNB 20:1, 0.03% solids, 8 μL/cm; CN95 | 1062-057 |
| 17B6 1 mg/mL & GAM IgG 2 mg/mL | |
| 8B10 CNB 40:1, 0.03% solids, 8 μL/cm; CN95 | 1062-057 |
| 17B6 1 mg/mL & GAM IgG 2 mg/mL | |
| 96-well plate, non-binding, Greiner bio-one | E20073CJ |
| P/N: 655901 | |
| 2% Tergitol buffer in 1xPBS | 24FEB2021CCB-2% |
| 55° C. Incubator Oven | OV009 |

Method: The stressed set of strips were placed at 55° C. for 72 hours before testing took place. A 96 well plate was set up with 80 μl of 2% tergitol buffer in 1×PBS. The strips were placed into each well and run vertically for 15 minutes. After 15 minutes, the tests were checked for nonspecific binding.

Discussion: This experiment showed that the tests had nonspecific binding in all conjugation concentration range when the lateral flow assay was placed at 55° C.

It was determined that with cellulose nanobeads, the concentration of the conjugate and/or test line antibody should be reduced.

### Example 8-Comparison of Conjugate Pads

The conjugate pad is the part of the lateral flow assay that contains strongly colored or fluorescent nanoparticles that have an antibody on their surface. When the liquid reaches the conjugate pad, these dried nanoparticles are released and mix with the sample. To test the effect of drying the conjugates (gold and CNBs) on the performance of the assay using various conjugate pads, the following experiments were performed.

### Materials and Equipment:

Description

Trichophyton Rubrum fungal extract, X612, 1 mg/mL
10 × PBS Fish Gel Concentrate
17B6.1E3 CN95 membrane, 1 mg/mL
13E10.1E2.1D4.1F3.E2 CN95 membrane, 1 mg/mL
8B10.1G6.1B5.1G3.D6 gold conjugate, OD 1.7
17B6.1E3 CNB conjugate, 0.22% solids
Tween 20
1X PBS, pH 7.4
1X PBS, 1% Casein, 0.5% Tween 20, 5% Sucrose, 2.5% trehalose
96-well plate, non-binding, Greiner bio-one P/N: 655901
Fusion 5, Cytiva formerly GE Healthcare, conjugate pad
Standard 17, Cytiva formerly GE Healthcare, conjugate pad
Grade 6614, Ahlstrom, conjugate pad
Grade 8951, Ahlstrom, conjugate pad
Grade 8980, Ahlstrom, conjugate pad
Ahlstrom 243 absorption pad, 21 mm
MIBA backing card, PN: MIBA-020
Biodot Cutter CM4000 CU001

Methods: The full strip is assembled by adhering the membrane to the backing card. The conjugate and absorption pads (overlap the membrane by +2 mm) on the bottom and top of the membrane respectively. The card was cut into 4 mm strips. 4 μl gold conjugate at OD 10 was added to the top end of the conjugate pads. 4 μl CNB conjugate at 0.03% solids was added to the top end of the conjugate pads (diluted in 1×PBS, 1% casein, 0.5% tween 20, 5% sucrose, 2.5% trehalose). The strips were then placed in an oven at 40° C. for 30 minutes.

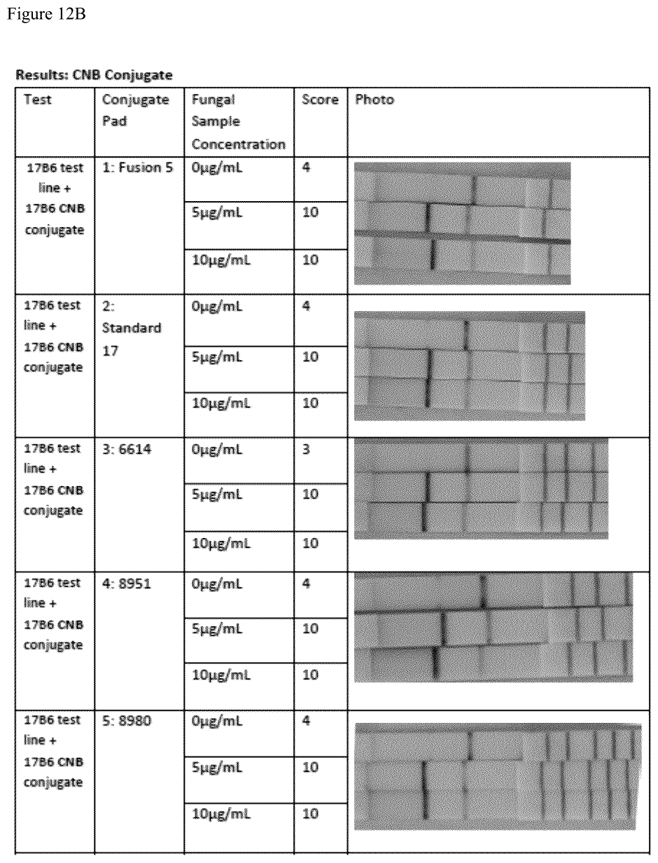

The fungal extract was diluted to 10 μg/mL and 5 μg/mL using 1× PB Fish Gel with 0.5% tween 20. A 96 well plate was set up as follows: 80μ L of the extract dilutions were added to each well. For the negative control, 80 μL of 1× PBS Fish Gel with 0.5% Tween 20 was used. The strips were placed into each well and run for 15 minutes. After 15 minutes, the tests were graded using the DCN grade scale from 0-10. The results are shown in FIG. 12A-B.

Discussion: All 5 gold conjugate pads worked well and released the gold efficiently. There was no nonspecific binding observed, and only a slight difference in sensitivity between the different pads. There was some gold aggregation observed underneath the conjugate pad and membrane overlap in pads 1, 2, and 5. Grade 6614 looked the cleanest.

Similarly to the gold conjugate, for cellulose nanobeads the grade 6614 pad showed less nonspecific binding compared to the other pads. However, the CNB conjugate resulted in NSB with all the test pads. Decreasing the percent solids could remove the NSB to allow comparison of sensitivity to the gold assay.

### Example 9-Testing Conjugates to Decrease Nonspecific Binding

This experiment tested conditions to eliminate nonspecific binding in assays using cellulose nanobeads.

### Materials and Equipment:

| Description | Lot No. |
|---|---|
| 10 × PBS Fish Gel Concentrate | 45117 |
| 17B6.1E3 CN95 membrane, 1 mg/mL | 1062-006 |
| 17B6.1E3 CNB conjugate, 0.22% solids | 1062-012 |
| Tween 20 | SLCC6187 |
| 1X PBS, pH 7.4 | W21-054 |
| 1X PBS, 1% Casein, 0.5% Tween 20, 5% Sucrose, | 22FEB2021CCB |
| 2.5% trehalose | |
| 96-well plate, non-binding, Greiner bio-one | E20073CJ |
| P/N: 655901 | |
| Grade 6614, Ahlstrom, conjugate pad | 002371 |
| Ahlstrom 243 absorption pad, 21 mm | 113701 |
| MIBA backing card, PN: MIBA-020 | 076026-02 |
| Biodot Cutter CM4000 CU001 | NA |

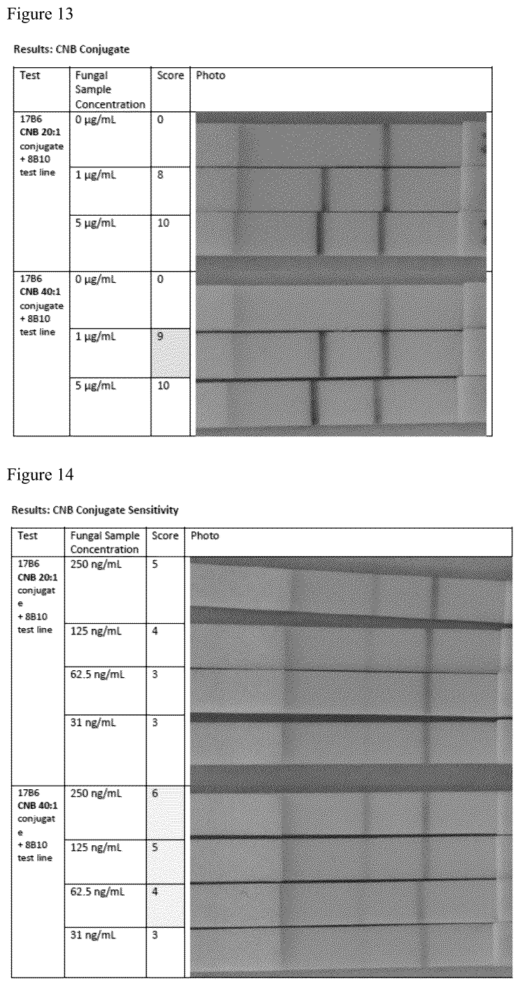

Methods: The full strip was assembled and the conjugates were applied as previously described. 4 μl CNB conjugate at 0.015% and 0.0075% solids were added to a grade 6614 conjugate pad. A 96 well plate was set up as follows: either 80 μl of 1×PBS Fish gel with 0.5% Tween 20 or 80 μl was 1× PBS was added to the wells. The strips were placed into each well, and allowed to run for 15 minutes. After 15 minutes, the tests were graded using the DCN grade scale from 0-10. The results are shown in FIG. 13.

Discussion: Decreasing the CNB concentration from 0.03% solids to 0.015% and 0.0075% did not remove the nonspecific binding. Further, the 1× PBS did not exhibit nonspecific binding, indicating the cause of the nonspecific binding is the fish gelatin or the Tween 20. Based on this, the 1× PBS fish gel may be eliminated from fungal sample from nails.

An experiment was also performed to test the effect of antibody concentration on the development of nonspecific binding in the assay using CNB as a conjugate. For this experiment, the 17B6 mAb was used, and 1× PBS with 2% tergitol was used as the nail sample buffer.

### Materials and Equipment:

| Description | Lot No. |
|---|---|
| 8B10 CN95 membrane, 1 mg/mL | 1062-016 |
| 17B6.1E3 CNB conjugate, 20:1, 0.20% solids | 1062-024 |

### Materials and Equipment:

| Description | Lot No. |
|---|---|
| 17B6.1E3 CNB conjugate, 40:1, 0.20% solids | 1062-024 |
| 2% Tergitol sample buffer | 24FEB2021CCB-2% |
| 25 mM Borate, 3% Casein, 0.5% Tween 20, 5% | 25FEB2021CCB |
| Sucrose, 2.5% trehalose | |
| 96-well plate, non-binding, Greiner bio-one | E20073CJ |
| P/N: 655901 | |
| Grade 6614, Ahlstrom, conjugate pad | 002371 |
| Ahlstrom 243 absorption pad, 21 mm | 113701 |
| MIBA backing card, PN: MIBA-020 | 076026-02 |
| Biodot Cutter CM4000 CU001 | NA |
| Sonicator QSonica, SN003 | NA |

The CNB conjugate was diluted at 20:1 and 40:1 in CNB dilution buffer (25 mM Borate, 3% casein, 0.5% Tween 20, 5% sucrose, and 1.5% trehalose). The 0.20% solid CNB conjugations were sonicated before being diluted to 0.03% in the dilution buffer (e.g. 7.5 μl CNB in 42.5 μl dilution buffer). 4 μl of the CNB conjugate at 0.03% solids were added to the grade 6614 conjugate pad and dried for 30 minutes as 40° C.

The fungal extract dilution buffer was prepared as indicated:

| | | 2% |
|---|---|---|
| | Fungal | Tergitol |
| | Extract | Sample |
| | (1 mg/mL) | Buffer |
| 0 μg/mL | 0 μL | 5000 μL |
| 1 μg/mL | 0.5 μL | 499.5 μL |
| 5 μg/mL | 2.5 μL | 497.5 μL |

A 96 well plate was set up as follows: 80 μl of sample was added to the wells. The strips were placed into each well, and allowed to run for 15 minutes. After 15 minutes, the tests were graded using the DCN grade scale from 0-10. The results are shown in FIG. 13.

Discussion: Decreasing the CNB loading from 10:1 down to 20:1 and 40:1 removed the nonspecific binding observed with higher concentrations. Initial results indicated that a dilution of 40:1 may be slightly more sensitive than the 20:1 dilution.

To test the sensitivity of the 20:1 and 40:1 CNB conjugates, the assay was repeated with fungal extracts ranging from 250 ng/ml to 31 ng/mL in 2% tergitol sample buffer.

| | | Fungal Extract | | 2% Tergitol | |
|---|---|---|---|---|---|
| | | (1 μg/mL) | | Sample Buffer | |
| 250 | ng/mL | 125 | μL | 375 | μL |
| 125 | ng/mL | 62.5 | μL | 437.5 | μL |
| 62.5 | ng/mL | 31.25 | μL | 468.75 | μL |
| 31 | ng/mL | 15.5 | μL | 484.50 | μL |

The 96 well plate was set up and the assay performed as described previously. The results are shown in FIG. 14.

Discussion: Both conjugation conditions detected fungal extracts down to 31 ng/ml. The 40:1 condition showed better sensitivity in the upper ranges. The use of less mAb coupled with the fact that nonspecific binding was seen at 10:1 CNB suggest that the 40:1 loading condition should be studied. Using tergitol at 2% did not adversely affect the CNB assay, and was tested using gold as the conjugate instead of CNB.

To test the effect of different concentrations of tergitol on the sensitivity of the assay using gold as the conjugate, the assay was run using tergitol at concentrations between 2% and 10%.

### Materials and Equipment:

| Description | Lot No. |
|---|---|
| 13E10.1E2.1D4.1F3.E2 CN95 membrane, | 1062-016 |
| 1 mg/mL | |
| 8B10 Gold Conjugate, pH 7.0, 20.0 μg/mL | 1062-019 |
| Ab load, 10.8 OD/mL | |
| 17B6 and anti-mouse IgG striped CN95, 1 mg/mL | 1062-016 |
| 1xPBS, pH 7.4 | W21-054 |
| Tergitol (70% in water) | MKCD6607 |
| Fungal Extract 1 mg/mL | 336803 |
| Ahlstrom 243 absorption pad, 21 mm | 113701 |
| MIBA backing card, PN: MIBA-020 | 076026-02 |
| 96-well plate, non-binding, Greiner bio-one | E20073CJ |
| P/N: 655901 | |
| 2% Tergitol sample buffer | 24FEB2021CCB-2% |
| 5% Tergitol sample buffer | 24FEB2021CCB-5% |
| 10% Tergitol sample buffer | 24FEB2021CCB- |
| | 10% |

The tergiotol sample buffer was diluted as indicated:

| | Tergitol (70% in | 1xPBS, |
|---|---|---|
| | water) | pH 7.4 |
| 2% Tergitol | 114 μL | 3886 μL |
| 5% Tergitol | 286 μL | 3714 μL |
| 10% Tergitol | 572 μL | 3428 μL |

The fungal extract was diluted as indicated:

| | | Tergitol |
|---|---|---|
| | Fungal | Buffer |
| | Extract | Sample (2, |
| | (1 mg/mL) | 5 & 10%) |
| 0 μg/mL | 0 μL | 5000 μL |
| 1 μg/mL | 0.5 μL | 499.5 μL |
| 5 μg/mL | 2.5 μL | 497.5 μL |

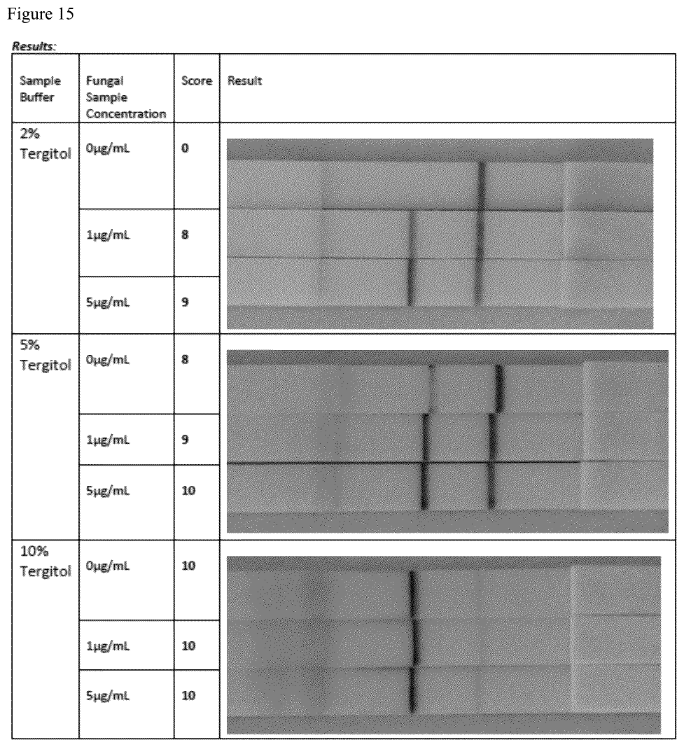

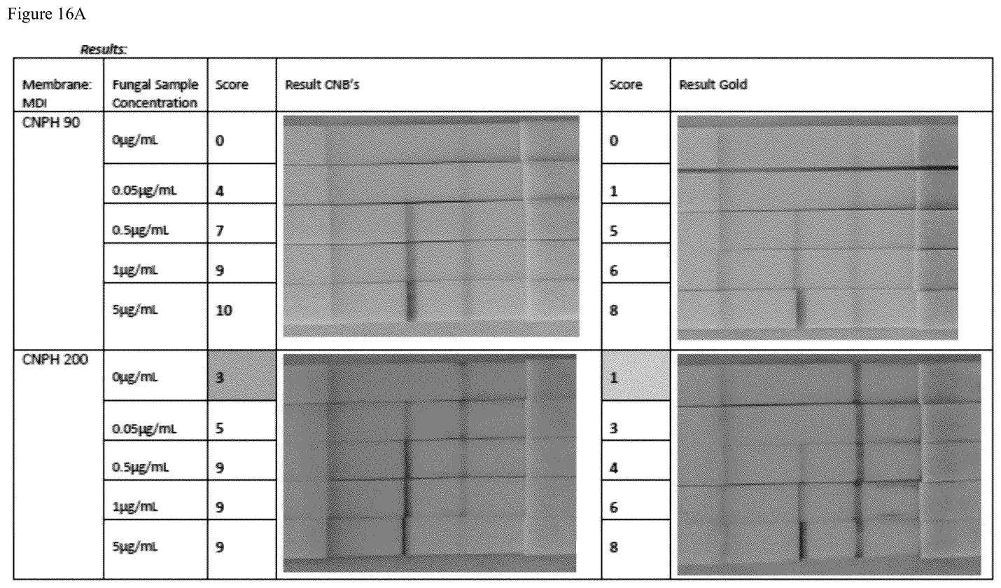

The 96 well plate was set up with 80 μl of sample in each well and the assay performed as described previously. The results are shown in FIG. 15.

Discussion: While the 2% tergitol showed no nonspecific binding, both 5% and 10% tergitol caused the gold conjugate to bind nonspecifically, to the point where at 10%, there was very little conjugate left to bind at the control line. Further studies demonstrated that, compared with gold conjugates, cellulose nanobeads (CNBs) showed less nonspecific binding with increased tergitol concentrations (data not shown). However, nonspecific binding was observed at 5% and 10% tergitol. It was also observed that increasing tergitol concentrations reduced the release of the CNBs from the conjugate pad, and 2% tergitol caused the control line to be weaker. Tergitol concentrations of 2%, 2.5%, 3%, 3.5%, 4%, and 4.5% were tested (data not shown). Very slight nonspecific binding was observed with the 4% and 4.5% tergitol concentrations using CNBs as the conjugate.

Further experiments demonstrated that for CNBs, both 0.06% and 0.09% solids caused no nonspecific binding (data not shown). There was higher background staining of the 0.09% membrane, suggesting that this concentration of CNBs starts to show aggregation.

With the gold conjugate, OD20 and OD30 both showed some nonspecific binding in 2% tergitol sample buffer (data not shown). Slight nonspecific binding was observed at OD12, with higher levels observed at OD14, OD16, and OD18 (data not shown). The concentration of the gold conjugate should thus be no more than OD10 to prevent non-specific binding.

### Example 10-Optimization of Nitrocellulose Membranes

The performance of various nitrocellulose membranes in the lateral flow assay was compared. These membranes differ in proprietary blend of chemicals added by the manufacturer and the physical pore size of each membrane. These variables can affect the performance of a test (e.g. slow v fast flow rate).

### Materials and Equipment:

| Description | Lot No. |
|---|---|
| CN95, Sartorius, 1 mg/mL 17B6, 1 mg/mL GAM IgG | 1062-031 |
| CN140, Sartorius, 1 mg/mL 17B6, 1 mg/mL GAM IgG | 1062-031 |
| FF120Plus, Whatman, 1 mg/mL 17B6, 1 mg/mL GAM IgG | 1062-031 |
| FF170Plus, Whatman, 1 mg/mL 17B6, 1 mg/mL GAM IgG | 1062-031 |
| 90CNPH-N-SS40, MDI, 1 mg/mL 17B6, 1 mg/mL GAM IgG | 1062-031 |
| 200CNPH-N-SS60, MDI, 1 mg/mL 17B6, 1 mg/mL GAM IgG | 1062-031 |
| 17B6.1E3 Gold conjugate (OD 10.5), 8 μL/cm | 1062-023 |
| 17B6.1E3 CNB conjugate (40:1), 0.20% solids, 8 μL/cm | 1062-032 |
| Fungal Extract 1 mg/mL | 336803 |
| Ahlstrom 243 absorption pad, 21 mm | 113701 |
| MIBA backing card, PN: MIBA-020 | 076026-02 |
| 9&-well plate, non-binding, Greiner bio-one P/N: 655901 | E20073CJ |
| 2% Tergitol in 1xPBS sample buffer | 24FEB2021CCB-2% |

Method: The nitrocellulose membrane was adhered onto the center portion of the backing card and striped with the 17B6 antibody. The conjugate/sample pad and absorption pad were adhered on either side of the membrane (overlap by +2 mm) and cut into 4 mm strips. The conjugate pads were sprayed with either gold or CNBs conjugated to the 17B6 antibody. Fungal extracts were diluted as indicated:

| | | Fungal Extract | | Tergitol Sample | |
|---|---|---|---|---|---|
| | | (1 mg/mL)(*5 μg/mL) | | Buffer | |
| 0 | μg/mL | 0 | μL | 5000 | μL |
| 0.05 | μg/mL | *50 | μL | 4950 | μL |
| 0.5 | μg/mL | 2.5 | μL | 4997.5 | μL |

| | | | |
|---|---|---|---|
| 1 | μg/mL | 5 | 4995 |
| 5 | μg/mL | 25 | 4975 |

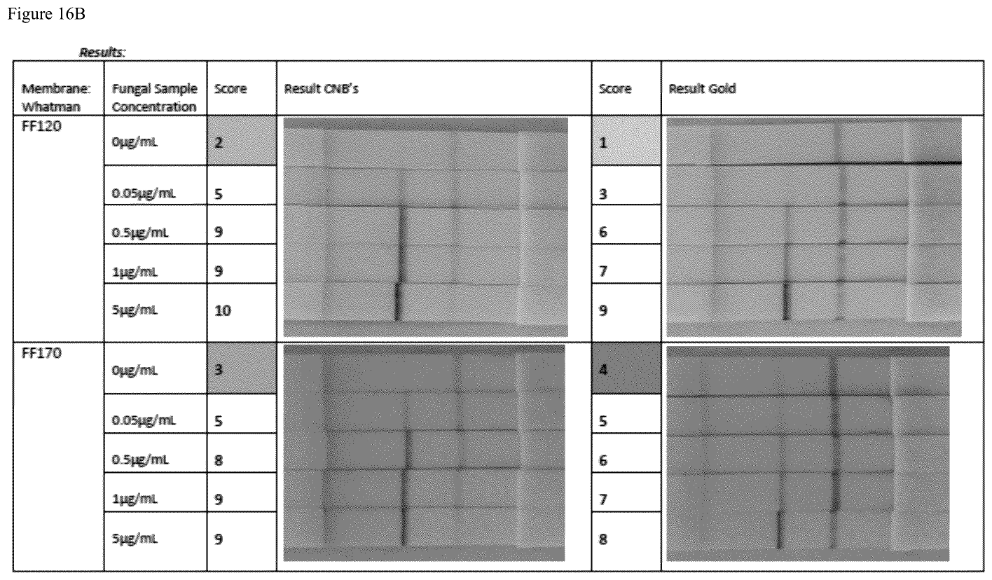

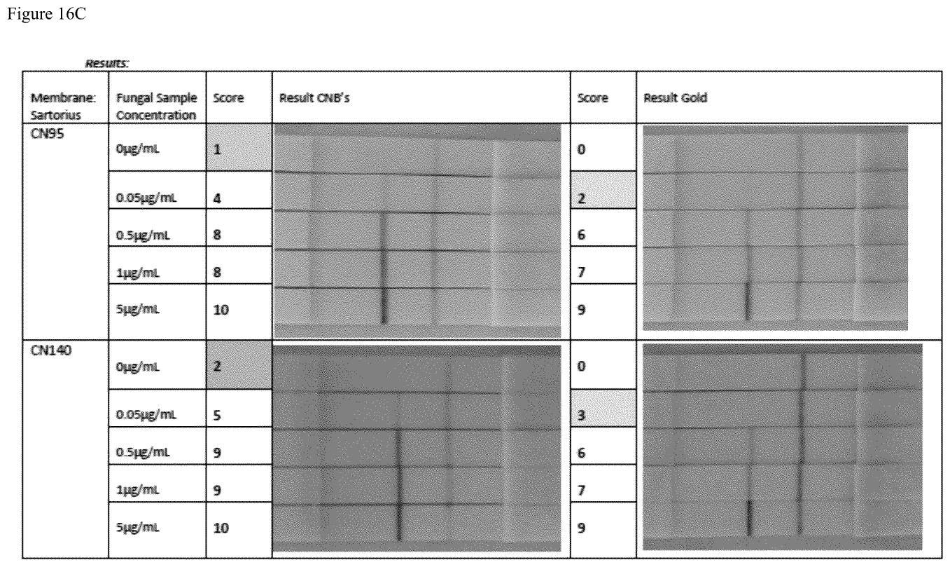

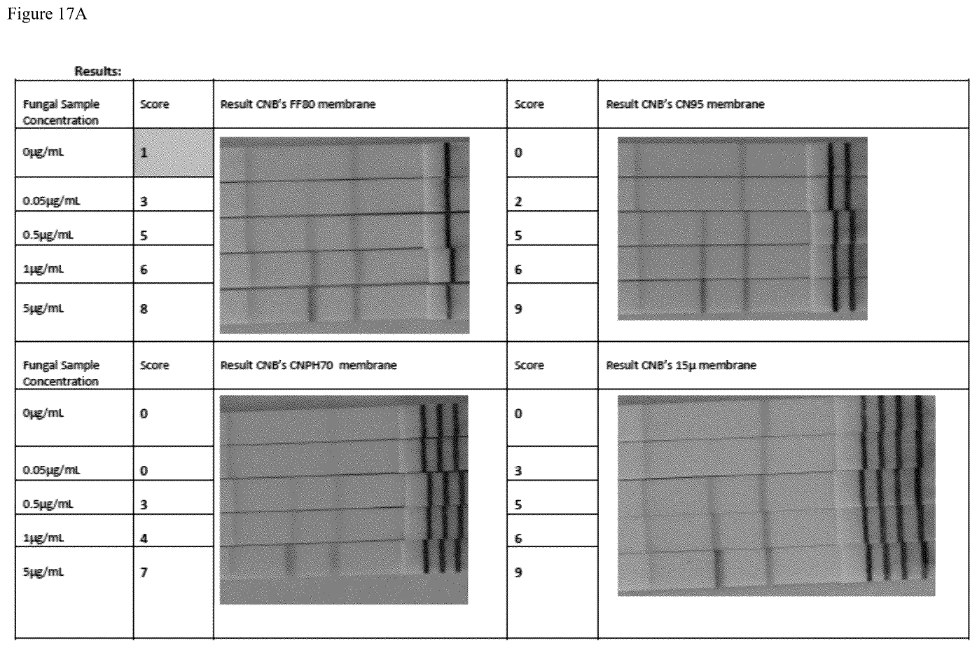

The 96 well plate was set up with 80 μl of sample in each well and the assay performed as described previously. The results are shown in FIG. 16A-C.

Discussion: The MDI CNPH 90 membrane worked for both the CNB and gold conjugates, however the control line was noticeably weak with both conjugates. The lines also spread out when striping, which could account for a weaker signal intensity. The MDI CNPH 200 membrane, which has small pores and hence flows slower, did not perform well for either conjugate. It was observed that the conjugates battled to clear the membrane in 15 minutes, and there was significant nonspecific binding. The Whatman FF120 membrane worked relatively well for the CNB conjugate, but some nonspecific binding was observed. However, none of the tests run with the Whatman membrane showed optimal flow, and the line quality was poor. This membrane likely requires blocking post striping to aid in conjugate-sample flow. The Sartorius CN95 membrane worked well with both conjugates with the result window being clean and the line quality good. The Sartorius CN140 worked similarly to the CN95 for the gold conjugate, with a slight increase in sensitivity at 0.05 μg/mL. If the test must be run in a short period of time, the CN95 membrane may be the best to use. The Sartorius CN140 cause nonspecific binding and background staining with the CNB conjugate, which appears to prefer faster membranes.

This experiment was repeated with additional membranes and using dual antibody pairs: Materials and Equipment:

| Description | Lot No. |
|---|---|
| FF80Plus, Whatman, 1 mg/mL 8B10, 1 mg/mL GAM IgG | 1062-036 |
| CN95, Sartorius, 1 mg/mL 8B10, 1 mg/mL GAM IgG | 1062-036 |
| CNPH70, MDI, 1 mg/mL 8B10, 1 mg/mL GAM IgG | 1062-036 |
| 15μ, MDI, 1 mg/mL 8B10, 1 mg/mL GAM IgG | 1062-036 |
| CN95, Sartorius, 1 mg/mL 13E10, 1 mg/mL GAM IgG | 1062-036 |
| CN150, Sartorius, 1 mg/mL 13E10, 1 mg/mL GAM IgG | 1062-036 |
| 8μ, MDI, 1 mg/mL 13E10, 1 mg/mL GAM IgG | 1062-036 |
| 8B10 Gold conjugate (OD 10.8), 8 μL/cm | 1062-023 |
| 17B6.1E3 CNB conjugate (40:1), 0.20% solids, 8 μL/cm | 1062-032 |
| Fungal Extract 1 mg/mL | 336803 |
| Ahlstrom 243 absorption gad, 21 mm | 113701 |
| MIBA backing card, PN: MIBA-020 | 076026-02 |
| 96-well plate, non-binding, Greiner bio-one P/N: 655901 | E20073CJ |
| 2% Tergitol in 1xPBS sample buffer | 24FEB2021CCB-2% |

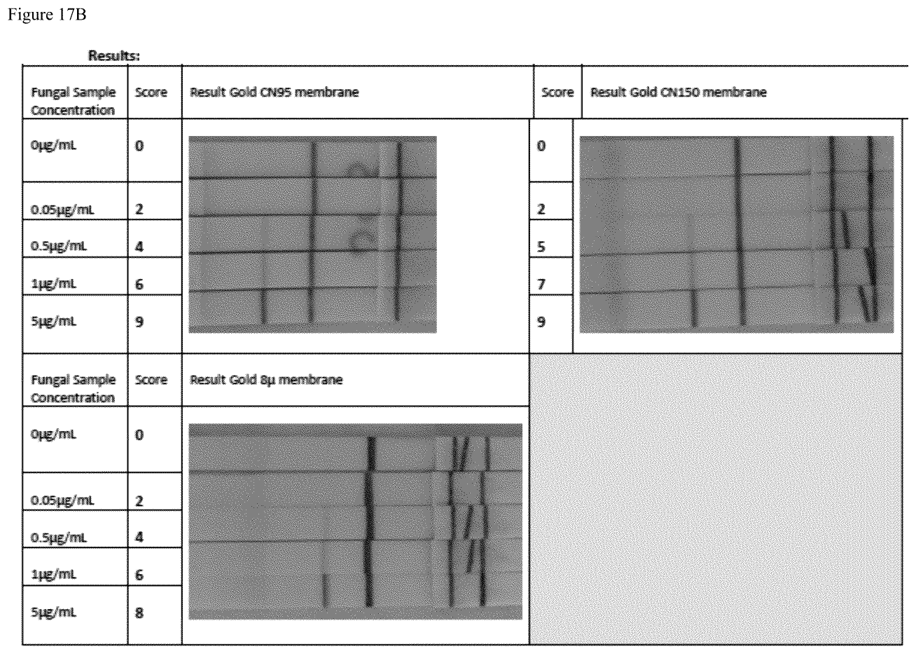

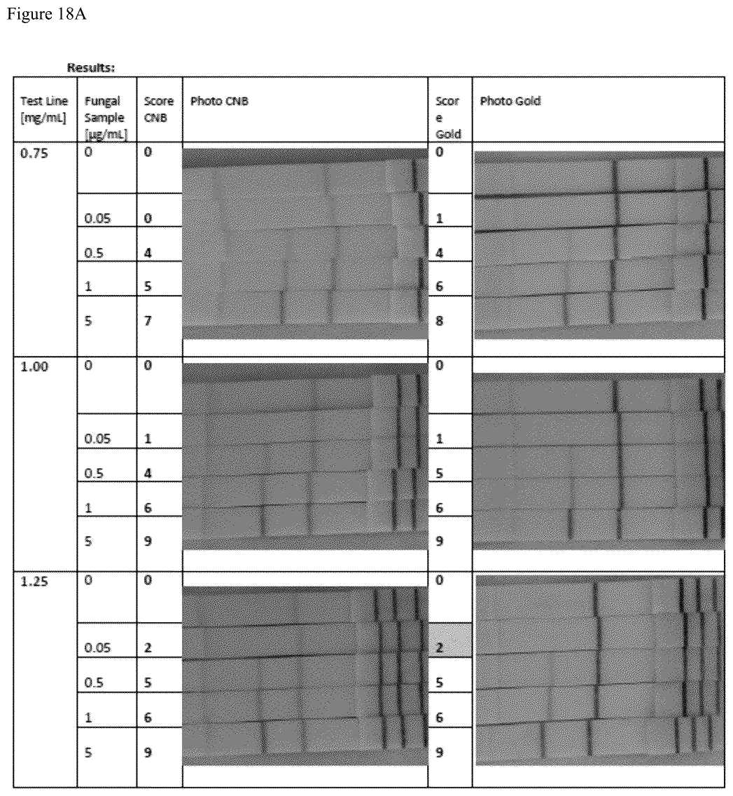

The results are demonstrated in FIG. 17A-B.

Discussion: For the cellulose nanobeads, the best performing membrane is the CN95. Using dual-pair antibodies removed the nonspecific binding seen previously, although the sensitivity was lower. The MDI 15μ membrane worked well, but the line quality was poor. The CNPH70 membrane resulted in wide lines that were noticeably weaker in intensity compared to the others. The Whatman FF80 showed slight nonspecific binding and poor line quality.

For the gold conjugate assay, the CN150 worked better than the CN95, however the CN150 and the MDI 8 μmembranes resulted in aggregation of the conjugate at the conjugate pad/membrane interphase.

### Example 11-Test Line Antibody Concentrations

This experiment tested the sensitivity and specificity of varied concentrations of the test line antibodies in both CNB and gold conjugate assays.

### Materials and Equipment:

| Description | Lot No. |
|---|---|
| CN95 membrane striped with [varying concentrations] 13E10 | 1062-043 |
| and 1 mg/mL GAM IgG | |
| CN95 membrane striped with [varying concentrations] 8B10 | 1062-043 |
| and 1 mg/mL GAM IgG | |
| 8B10 gold conjugate (OD10~diluted the OD20 down and sprayed 8 μL/cm) | 1062-039 |
| 17B6 CNB conjugate (40:1, 0.03% solids and sprayed 8 μL/cm) | 1062-032 |
| Ahlstrom 243 absorption pad, 21 mm | 113701 |
| MIBA backing card, PN: MIBA-020 | 076026-02 |
| 96-well plate, non-binding, Greiner bio-one P/N:655901 | E20073CJ |
| 2% Tergitol in 1xPBS sample buffer | 24FEB2021CCB-2% |
| Trichophyton Rubrum fungal extract, X612, 1 mg/ml | 336803 |

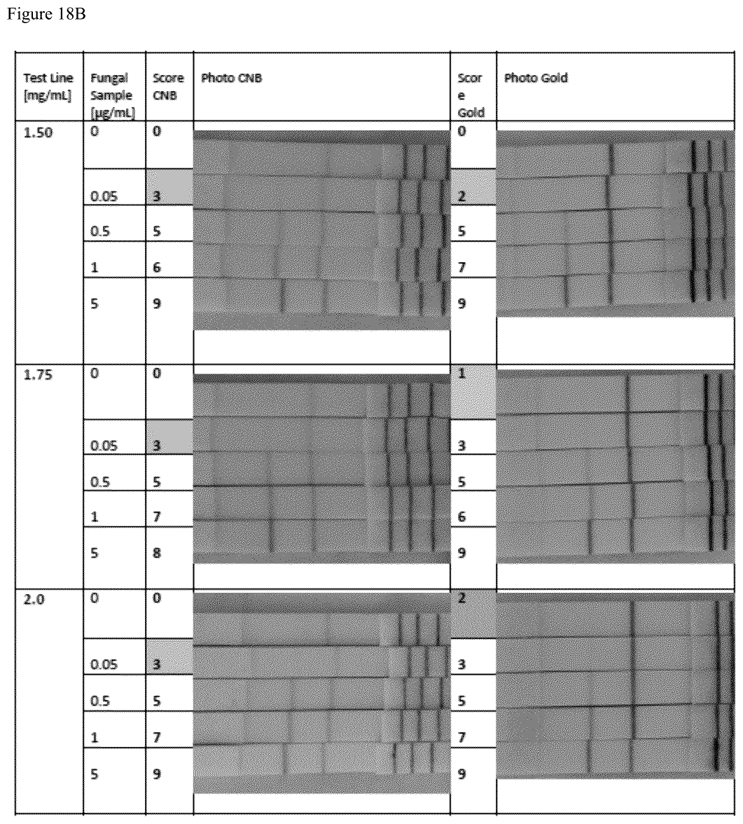

Test strips were and assays were run as previously described. The results are shown in FIG. 18A-B.

Discussion: CNBs did not result in any nonspecific binding for any of the test conditions up to 2 mg/mL 8B10 antibody. The best sensitivity was observed at 1.50 mg/mL, and from there on, sensitivity plateaued. The best sensitivity for the gold assay was observed at 1.25 mg/mL and 1.50 mg/mL; nonspecific binding stated being observed at 1.75 and 2.0 mg/mL.

### Numbered Embodiments

Other subject matter contemplated by the present disclosure is set out in the following numbered embodiments:
Embodiment 1. A method of differentially detecting one or more dermatophyte organisms in a sample, said method comprising:
   a) collecting a sample from a subject;
   b) incubating the subject sample in a sample buffer comprising a non-ionic detergent at room temperature;
   c) applying the sample in the sample buffer to a lateral flow assay, wherein the lateral flow assay comprises:
      i) a nitrocellulose membrane comprising one or more unconjugated capture antibodies that binds to an dermatophyte antigen;
      ii) a conjugate pad comprising one or more detection antibodies comprising an antibody-nanoparticle conjugate that binds to a dermatophyte antigen;
      iii) a control antibody that binds to the unconjugated nanoparticle;
   d) allowing the lateral flow assay to run for at least 10 minutes;
   e) reading the test display to determine whether one or more dermatophyte organisms are present in the subject sample.
Embodiment 2. The method of embodiment 1, wherein the one or more capture antibodies and one or more detection antibodies bind to the same antigen.
Embodiment 3. The method of embodiment 1 or embodiment 2, wherein the one or more capture antibodies and one or more detection antibodies bind to different antigens on the same dermatophyte organism.
Embodiment 4. The method of any of embodiments 1-3, wherein the one or more capture and detection antibodies are different antibodies.
Embodiment 5. The method of any of embodiments 1-3, wherein the one or more capture and detection antibodies are the same antibodies.
Embodiment 6. The method of any of embodiments 1-5, wherein the one or more capture antibodies or one or more detection antibodies comprise a VL amino acid sequence at least 70% identical to any one of SEQ ID NO: 4, SEQ ID NO: 8, or SEQ ID NO: 12.
Embodiment 7. The method of any of embodiments 1-6, wherein the one or more capture antibodies or one or more detection antibodies comprise a VH amino acid sequence at least 70% identical to any one of SEQ ID NO: 3, SEQ ID NO: 7, or SEQ ID NO: 11.
Embodiment 8. The method of any of embodiment s 1-7, wherein the one or more capture antibodies or one or more detection antibodies comprise VH amino acid sequence pair at least 70% identical to any one of SEQ ID NO: 3, SEQ ID NO: 7, or SEQ ID NO: 11, and VL amino acid sequence at least 70% identical to any one of SEQ ID NO: 4, SEQ ID NO: 8, or SEQ ID NO: 12.
Embodiment 9. The method of any of embodiments 1-8, wherein the one or more capture antibodies or one or more detection antibodies comprise:
a) a VH amino acid sequence at least 70% identical SEQ ID NO: 3 and a VL amino acid sequence at least 70% identical to SEQ ID NO: 4;
b) a VH amino acid sequence at least 70% identical to SEQ ID NO: 7 and a VL amino acid sequence at least 70% identical to SEQ ID NO: 8; or
c) a VH amino acid sequence at least 70% identical to SEQ ID NO: 11 and a VL amino acid sequence at least 70% identical to SEQ ID NO: 12.
Embodiment 10. The method of any of embodiments 1-9, wherein the one or more detection antibodies is conjugated to colloidal gold or a cellulose nanobcad.
Embodiment 11. The method of any of embodiments 1-10, wherein the one or more detection antibodies are conjugated to different cellulose nanobeads that provide different read-outs, allowing the detection and differentiation of at least two different dermatophyte organisms.
Embodiment 12. The method of any of embodiment s 1-11, wherein one or more capture or one or more detection antibodies do not bind to non-dermatophyte organisms in the subject sample.
Embodiment 13. The method of any of embodiment s 1-12, wherein the one or more capture or one or more detection antibodies do not bind to a yeast.
Embodiment 14. The method of embodiment 13, wherein the yeast is a Candida.
Embodiment 15. The method of any of embodiments 1-14, wherein the one or more dermatophyte organisms are Trichophyton fungi, Microsporum fungi, or Epidermophyton fungi.
Embodiment 16. The method of any of embodiments 1-15, wherein the one or more dermatophyte organisms are Microsporum amazonicum, Microsporum audouinii, Microsporum boullardii, Microsporum canis, Microsporum canis var. distortum, Microsporum cookci, Microsporum distortum, Microsporum duboisii, Microsporum equinum, Microsporum ferrugineum, Microsporum fulvum, Microsporum gallinac, Microsporum gypseum, Microsporum langeronii, Microsporum nanum, Microsporum persicolor, Microsporum praecox, Microsporum ripariac, Microsporum rivalieri, Epidermophyton floccosum, Epidermophyton stockdaleae, Trichophyton benhamiac, Trichophyton bullosum, Trichophyton concentricum, Trichophyton equinum, Trichophyton eriotrephon, Trichophyton erinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, andTrichophyton violaceum.
Embodiment 17. The method of any one of embodiments 1-16, wherein the one or more dermatophyte organisms are T. rubrum, T. interdigitale, E. floccosum, or M. canis.
Embodiment 18. A lateral flow assay for the differential detection of one or more dermatophyte organisms comprising:
   a) a nitrocellulose membrane comprising one or more unconjugated capture antibodies that binds to an dermatophyte antigen;
   b) a conjugate pad comprising one or more detection antibodies comprising an antibody-nanoparticle conjugate that binds to a dermatophyte antigen;
   c) a control antibody that binds to the unconjugated nanoparticle.
Embodiment 19. The lateral flow assay of embodiment 18, wherein the one or more capture antibodies and one or more detection antibodies bind to the same antigen.
Embodiment 20. The lateral flow assay of embodiment 18 or embodiment 19, wherein the one or more capture antibodies and one or more detection antibodies bind to different antigens on the same dermatophyte organism.
Embodiment 21. The lateral flow assay of any of embodiments 18-20, wherein the one or more capture and detection antibodies are different antibodies.
Embodiment 22. The lateral flow assay of any of claims 18-21, wherein the one or more capture and detection antibodies are the same antibodies.
Embodiment 23. The lateral flow assay of any of embodiments 18-22, wherein the one or more capture antibodies or one or more detection antibodies comprise a VL amino acid sequence at least 70% identical to any one of SEQ ID NO: 4, SEQ ID NO: 8, or SEQ ID NO: 12.
Embodiment 24. The lateral flow assay of any of embodiments 18-23, wherein the one or more capture antibodies or one or more detection antibodies comprise a VH amino acid sequence at least 70% identical to any one of SEQ ID NO: 3, SEQ ID NO: 7, or SEQ ID NO: 11.
Embodiment 25. The lateral flow assay of any of embodiments 18-24, wherein the one or more capture antibodies or one or more detection antibodies comprise VH amino acid sequence pair at least 70% identical to any one of SEQ ID NO: 3, SEQ ID NO: 7, or SEQ ID NO: 11, and VL amino acid sequence at least 70% identical to any one of SEQ ID NO: 4, SEQ ID NO: 8, or SEQ ID NO: 12.
Embodiment 26. The lateral flow assay of any of embodiments 18-25, wherein the one or more capture antibodies or one or more detection antibodies comprise:
   a) a VH amino acid sequence at least 70% identical SEQ ID NO: 3 and a VL amino acid sequence at least 70% identical to SEQ ID NO: 4;
   b) a VH amino acid sequence at least 70% identical to SEQ ID NO: 7 and a VL amino acid sequence at least 70% identical to SEQ ID NO: 8; or
   c) a VH amino acid sequence at least 70% identical to SEQ ID NO: 11 and a VL amino acid sequence at least 70% identical to SEQ ID NO: 12.
Embodiment 27. The lateral flow assay of any of embodiments 18-26, wherein the one or more detection antibodies is conjugated to colloidal gold or a cellulose nanobead.
Embodiment 28. The lateral flow assay of any of embodiments 18-27, wherein the one or more detection antibodies are conjugated to different cellulose nanobeads that provide different read-outs, allowing the detection and differentiation of at least two different dermatophyte organisms.
Embodiment 29. The lateral flow assay of any of embodiments 18-28, wherein one or more capture or one or more detection antibodies do not bind to non-dermatophyte organisms in the subject sample.
Embodiment 30. The lateral flow assay of any of embodiments 18-29, wherein the one or more capture or one or more detection antibodies do not bind to a yeast.
Embodiment 31. The lateral flow assay of embodiment 18, wherein the yeast is a Candida.
Embodiment 32. The lateral flow assay of any of embodiments 18-31, wherein the one or more dermatophyte organisms are Trichophyton fungi, Microsporum fungi, or Epidermophyton fungi.
Embodiment 33. The lateral flow assay of any of embodiments 18-32, wherein the one or more dermatophyte organisms are Microsporum amazonicum, Microsporum audouinii, Microsporum boullardii, Microsporum canis, Microsporum canis var. distortum, Microsporum cookci, Microsporum distortum, Microsporum duboisii, Microsporum equinum, Microsporum ferrugineum, Microsporum fulvum, Microsporum gallinac, Microsporum gypseum, Microsporum langcronii, Microsporum nanum, Microsporum persicolor, Microsporum praccox, Microsporum ripariac, Microsporum rivalieri, Epidermophyton floccosum, Epidermophyton stockdaleac, Trichophyton benhamiac, Trichophyton bullosum, Trichophyton concentricum, Trichophyton cquinum, Trichophyton criotrephon, Trichophyton crinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, andTrichophyton violaceum.
Embodiment 34. The lateral flow assay of any of embodiments 18-33, wherein the one or more dermatophyte organisms are T. rubrum, T. interdigitale, E. floccosum, or M. canis.
Embodiment 35. The lateral flow assay of any of embodiments 18-34, wherein the nitrocellulose membrane is an Ahlstrom 6614 membrane, a CN95 membrane, a CN140 membrane, a FF120 Plus membrane, a FF170 Plus membrane, a 90-CNPH-N-SS40 membrane, a 200CNPH-N-SS60 membrane, a FF80 Plus membrane, a CNPH70 membrane, a CN150 membrane, a 15μ membrane, or an 8μ membrane. In some embodiments, the nitrocellulose membrane is an Ahlstrom 6614 membrane, a 90-CNPH-N CN95 membrane, or a CN140 membrane.
Embodiment 36. The lateral flow assay of any of embodiments 18-35, wherein the lateral flow assay uses gold nanoparticles for detection and the nitrocellulose membrane is CN95 or CN140.
Embodiment 37. The lateral flow assay of any of embodiments 18-36, wherein the nitrocellulose membrane comprises between about 0.1 mg/mL to about 5 mg/mL of the one or more capture antibodies.
Embodiment 38. The lateral flow assay of any of embodiments 18-37, wherein the nitrocellulose membrane comprises about 1.0 mg/mL of the one or more capture antibodies.
Embodiment 39. The lateral flow assay of any one of embodiments 18-38, wherein the conjugate pad comprises between about 1 μl/cm to about 20 μl/cm of the one or more detection antibodies.
Embodiment 40. The lateral flow assay of any one of embodiments 18-39, wherein the conjugate pad comprises about 8 μl/cm of the one or more detection antibodies.
Embodiment 41. The lateral flow assay of any one of embodiments 18-40, wherein the conjugate pad comprises about 8 μl/cm of an antibody-gold conjugate.
Embodiment 42. The lateral flow assay of any one of embodiments 18-41, wherein the lateral flow assay can detect one or more dermatophyte organisms in an extract of between about 20 ng/ml to about 500 ng/mL.
Embodiment 43. The lateral flow assay of any one of embodiments 18-42, wherein the lateral flow assay is stable for at least 5 years.
Embodiment 44. The lateral flow assay of any one of embodiments 18-43, wherein the lateral flow assay is stable for at least 5 years at room temperature.
Embodiment 45. The lateral flow assay of any one of embodiments 18-44, wherein the lateral flow assay is stable for at least 300 days at room temperature with no significant decrease in sensitivity.
Embodiment 46. The lateral flow assay of any one of embodiments 18-45, wherein the lateral flow assay is stable for at least five years with refrigeration.
Embodiment 47. The lateral flow assay of any one of embodiments 18-46, wherein the sample buffer comprises a non-ionic or zwitterionic detergent.
Embodiment 48. The lateral flow assay of any one of embodiments 18-47, wherein the sample buffer is a digitonin or CHAPS buffer.
Embodiment 49. The lateral flow assay of any one of embodiments 18-48, wherein the sample buffer is a 1% digitonin or 1.5% CHAPS buffer.
Embodiment 50. An immunohistochemistry (IHC) assay for the differential detection of one or more dermatophyte organisms comprising:
   a) one or more unconjugated capture antibodies that binds to an dermatophyte antigen; and
   b) one or more detection antibodies comprising an antibody-nanoparticle conjugate that binds to a dermatophyte antigen;
Embodiment 51. The immunohistochemistry assay of embodiment 50, wherein the one or more capture antibodies and one or more detection antibodies bind to the same antigen.
Embodiment 52. The immunohistochemistry assay of embodiment 50 or embodiment 51, wherein the one or more capture antibodies and one or more detection antibodies bind to different antigens on the same dermatophyte organism.
Embodiment 53. The immunohistochemistry assay of any of embodiments 50-52, wherein the one or more capture and detection antibodies are different antibodies.
Embodiment 54. The immunohistochemistry assay of any of embodiments 50-53, wherein the one or more capture and detection antibodies are the same antibodies.
Embodiment 55. The immunohistochemistry assay of any of embodiments 50-54, wherein the one or more capture antibodies or one or more detection antibodies comprise a VL amino acid sequence at least 70% identical to any one of SEQ ID NO: 4, SEQ ID NO: 8, or SEQ ID NO: 12.
Embodiment 56. The immunohistochemistry assay of any of embodiments 50-55, wherein the one or more capture antibodies or one or more detection antibodies comprise a VH amino acid sequence at least 70% identical to any one of SEQ ID NO: 3, SEQ ID NO: 7, or SEQ ID NO: 11.
Embodiment 57. The immunohistochemistry assay of any of embodiments 50-56, wherein the one or more capture antibodies or one or more detection antibodies comprise VH amino acid sequence pair at least 70% identical to any one of SEQ ID NO: 3, SEQ ID NO: 7, or SEQ ID NO: 11, and VL amino acid sequence at least 70% identical to any one of SEQ ID NO: 4, SEQ ID NO: 8, or SEQ ID NO: 12.
Embodiment 58. The immunohistochemistry assay of any of embodiments 50-57, wherein the one or more capture antibodies or one or more detection antibodies comprise:
   a) a VH amino acid sequence at least 70% identical SEQ ID NO: 3 and a VL amino acid sequence at least 70% identical to SEQ ID NO: 4;
   b) a VH amino acid sequence at least 70% identical to SEQ ID NO: 7 and a VL amino acid sequence at least 70% identical to SEQ ID NO: 8; or
   c) a VH amino acid sequence at least 70% identical to SEQ ID NO: 11 and a VL amino acid sequence at least 70% identical to SEQ ID NO: 12.
Embodiment 59. The immunohistochemistry assay of any of embodiments 50-58, wherein the one or more detection antibodies is conjugated to colloidal gold or a cellulose nanobead.
Embodiment 60. The immunohistochemistry assay of any of embodiments 50-59, wherein the one or more detection antibodies are conjugated to different cellulose nanobeads that provide different read-outs, allowing the detection and differentiation of at least two different dermatophyte organisms.
Embodiment 61. The immunohistochemistry assay of any of embodiments 50-60, wherein one or more capture or one or more detection antibodies do not bind to non-dermatophyte organisms in the subject sample.
Embodiment 62. The immunohistochemistry assay of any of embodiments 50-61, wherein the one or more capture or one or more detection antibodies do not bind to a yeast.
Embodiment 63. The immunohistochemistry assay of embodiment 62, wherein the yeast is a Candida.
Embodiment 64. The immunohistochemistry assay of any of embodiments 50-63, wherein the one or more dermatophyte organisms are Trichophyton fungi, Microsporum fungi, or Epidermophyton fungi.
Embodiment 65. The immunohistochemistry assay of any of embodiments 50-64, wherein the one or more dermatophyte organisms are Microsporum amazonicum, Microsporum audouinii, Microsporum boullardii, Microsporum canis, Microsporum canis var. distortum, Microsporum cookei, Microsporum distortum, Microsporum duboisii, Microsporum equinum, Microsporum ferrugineum, Microsporum fulvum, Microsporum gallinae, Microsporum gypseum, Microsporum langeronii, Microsporum nanum, Microsporum persicolor, Microsporum praecox, Microsporum ripariae, Microsporum rivalieri, Epidermophyton floccosum, Epidermophyton stockdaleae, Trichophyton benhamiae, Trichophyton bullosum, Trichophyton concentricum, Trichophyton equinum, Trichophyton eriotrephon, Trichophyton erinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, and Trichophyton violaceum.
Embodiment 66. The immunohistochemistry assay of any of embodiments 50-66, wherein the one or more dermatophyte organisms are T. rubrum, T. interdigitale, E. floccosum, or M. canis.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.
Brillowska-Dabrowska, A., Saunte, D. M., & Arendrup, M. C. (2007 April). Five-hour diagnosis of dermatophyte nail infections with specific detection of Trichophyton rubrum. Journal of clinical microbiology.
Méhul, Bruno et al. “Detection of Trichophyton rubrum and Trichophyton interdigitale in onychomycosis using monoclonal antibodies against Sub6 (Tri r 2).” Mycoses vol. 62,1 (2019): 32-40. doi: 10.1111/myc .12843
Piraccini, B. M., & Alessandrini, A. (2015). Onychomycosis: A Review. Journal of fungi (Basel, Switzerland), 1 (1), 30-43.
Tchernev, G., P. K. Penev, P. Nenoff, L. G. Zisova, J. C. Cardoso, T. Taneva, G. Ginter-Hanslemayer, H. Ananiev, M. Gulubova, R. Hristova, D. Nocheva, C. Guarneri, G. Martino, & N. Kanazawa, (2013) Onychomycosis: Mondern diagnostic and treatment approaches, Wien Med Wochenschr. 163 (1-2): 1-12).

1. A method of differentially detecting one or more dermatophyte organisms in a sample, said method comprising:
a) collecting a sample from a subject;
b) incubating the subject sample in a sample buffer comprising a non-ionic detergent at room temperature;
c) applying the sample in the sample buffer to a lateral flow assay, wherein the lateral flow assay comprises:
i) a nitrocellulose membrane comprising one or more unconjugated capture antibodies that binds to an dermatophyte antigen;
ii) a conjugate pad comprising one or more detection antibodies comprising an antibody-nanoparticle conjugate that binds to a dermatophyte antigen;
iii) a control antibody that binds to the unconjugated nanoparticle;
d) allowing the lateral flow assay to run for at least 10 minutes;
e) reading the test display to determine whether one or more dermatophyte organisms are present in the subject sample.

2-7. (canceled)

8. The method of claim 1, wherein the one or more capture antibodies or one or more detection antibodies comprise VH amino acid sequence pair at least 70% identical to any one of SEQ ID NO: 3, SEQ ID NO: 7, or SEQ ID NO: 11, and VL amino acid sequence at least 70% identical to any one of SEQ ID NO: 4, SEQ ID NO: 8, or SEQ ID NO: 12.

9. The method of claim 1, wherein the one or more capture antibodies or one or more detection antibodies comprise:
a) a VH amino acid sequence at least 70% identical SEQ ID NO: 3 and a VL amino acid sequence at least 70% identical to SEQ ID NO: 4;
b) a VH amino acid sequence at least 70% identical to SEQ ID NO: 7 and a VL amino acid sequence at least 70% identical to SEQ ID NO: 8; or
c) a VH amino acid sequence at least 70% identical to SEQ ID NO: 11 and a VL amino acid sequence at least 70% identical to SEQ ID NO: 12.

10. The method of claim 1, wherein the one or more detection antibodies is conjugated to colloidal gold or a cellulose nanobead.

11. (canceled)

12. The method of claim 1, wherein one or more capture or one or more detection antibodies do not bind to non-dermatophyte organisms in the subject sample.

13-14. (canceled)

15. The method of claim 1, wherein the one or more dermatophyte organisms are Trichophyton fungi, Microsporum fungi, or Epidermophyton fungi.

16. The method of claim 1, wherein the one or more dermatophyte organisms are Microsporum amazonicum, Microsporum audouinii, Microsporum boullardii, Microsporum canis, Microsporum canis var. distortum, Microsporum cookei, Microsporum distortum, Microsporum duboisii, Microsporum equimim, Microsporum ferrugineum, Microsporum fulvum, Microsporum gallinae, Microsporum gypseum, Microsporum langeronii, Microsporum namm, Microsporum persicolor, Microsporum praecox, Microsporum ripariae, Microsporum rivalieri, Epidermophyton floccosum, Epidermophyton stockdaleae, Trichophyton benhamiae, Trichophyton bullosum, Trichophyton concentricum, Trichophyton equimim, Trichophyton eriotrephon, Trichophyton erinacei, Trichophyton interdigitale, Trichophyton mentagrophytes, Trichophyton quinckeamim, Trichophyton rubrum, Trichophyton schoenleinii, Trichophyton simii, Trichophyton soudanense, Trichophyton tonsurans, Trichophyton verrucosum, andTrichophyton violaceum.

17. The method of claim 1, wherein the one or more dermatophyte organisms are T. rubrum, T. interdigitale, E. floccosum, or M. canis.

18. A lateral flow assay for the differential detection of one or more dermatophyte organisms comprising:
a) a nitrocellulose membrane comprising one or more unconjugated capture antibodies that binds to an dermatophyte antigen;
b) a conjugate pad comprising one or more detection antibodies comprising an antibody-nanoparticle conjugate that binds to a dermatophyte antigen;
c) a control antibody that binds to the unconjugated nanoparticle.

19-24. (canceled)

25. The lateral flow assay of claim 18, wherein the one or more capture antibodies or one or more detection antibodies comprise VH amino acid sequence pair at least 70% identical to any one of SEQ ID NO: 3, SEQ ID NO: 7, or SEQ ID NO: 11, and VL amino acid sequence at least 70% identical to any one of SEQ ID NO: 4, SEQ ID NO: 8, or SEQ ID NO: 12.

26. The lateral flow assay of claim 18, wherein the one or more capture antibodies or one or more detection antibodies comprise:
a) a VH amino acid sequence at least 70% identical SEQ ID NO: 3 and a VL amino acid sequence at least 70% identical to SEQ ID NO: 4;
b) a VH amino acid sequence at least 70% identical to SEQ ID NO: 7 and a VL amino acid sequence at least 70% identical to SEQ ID NO: 8; or
c) a VH amino acid sequence at least 70% identical to SEQ ID NO: 11 and a VL amino acid sequence at least 70% identical to SEQ ID NO: 12.

27. The lateral flow assay of claim 18, wherein the one or more detection antibodies is conjugated to colloidal gold or a cellulose nanobead.

28. (canceled)

29. The lateral flow assay of claim 18, wherein one or more capture or one or more detection antibodies do not bind to non-dermatophyte organisms in the subject sample.

30-31. (canceled)

32. The lateral flow assay of claim 18, wherein the one or more dermatophyte organisms are Trichophyton fungi, Microsporum fungi, or Epidermophyton fungi.

33. The lateral flow assay of claim 18, wherein the one or more dermatophyte organisms are Microsporum amazonicum, Microsporum audouinii, Microsporum boullardii, Microsporum canis, Microsporum canis var. distortum, Microsporum cookei, Microsporum distortum, Microsporum duboisii, Microsporum equimim, Microsporum ferrugineum, Microsporum fulvum, Microsporum gallinae, Microsporum gypseum, Microsporum langeronii, Microsporum namm, Microsporum persicolor, Microsporum praecox, Microsporum ripariae, Microsporum rivalieri, Epidermophyton floccosum, Epidermophyton stockdaleae, Trichophyton benhamiae, Trichophyton bullosum, Trichophyton concentricum, Trichophyton equimim, Trichophyton eriotrephon, Trichophyton erinacei, Trichophyton interdigitale, Trichophyton mentagrophytes, Trichophyton quinckeamim, Trichophyton rubrum, Trichophyton schoenleinii, Trichophyton simii, Trichophyton soudanense, Trichophyton tonsurans, Trichophyton verrucosum, andTrichophyton violaceum.

34. The lateral flow assay of claim 18, wherein the one or more dermatophyte organisms are T. rubrum, T. interdigitale, E. floccosum, or M. canis.

35. The lateral flow assay of claim 18, wherein the nitrocellulose membrane is an Ahlstrom 6614 membrane, a CN95 membrane, a CN140 membrane, a FF120 Plus membrane, a FF 170 Plus membrane, a 90-CNPH-N-SS40 membrane, a 200CNPH-N-SS60 membrane, a FF80 Plus membrane, a CNPH70 membrane, a CN150 membrane, a 15μ membrane, or an 8μ membrane. In some embodiments, the nitrocellulose membrane is an Ahlstrom 6614 membrane, a 90-CNPH-N CN95 membrane, or a CN140 membrane.

36. The lateral flow assay of claim 18, wherein the lateral flow assay uses gold nanoparticles for detection and the nitrocellulose membrane is CN95 or CN140.

37. The lateral flow assay of claim 18, wherein the nitrocellulose membrane comprises between about 0.1 mg/mL to about 5 mg/mL of the one or more capture antibodies.

38. (canceled)

39. The lateral flow assay of claim 18, wherein the conjugate pad comprises between about 1 μl/cm to about 20 μl/cm of the one or more detection antibodies.

40.-41. (canceled)

42. The lateral flow assay of claim 18, wherein the lateral flow assay can detect one or more dermatophyte organisms in an extract of between about 20 ng/mL to about 500 ng/mL.

43.-49. (canceled)

50. An immunohistochemistry (IHC) assay for the differential detection of one or more dermatophyte organisms comprising:
a) one or more unconjugated capture antibodies that binds to an dermatophyte antigen; and
b) one or more detection antibodies comprising an antibody-nanoparticle conjugate that binds to a dermatophyte antigen;

51-56. (canceled)

57. The immunohistochemistry assay of claim 50, wherein the one or more capture antibodies or one or more detection antibodies comprise VH amino acid sequence pair at least 70% identical to any one of SEQ ID NO: 3, SEQ ID NO: 7, or SEQ ID NO: 11, and VL amino acid sequence at least 70% identical to any one of SEQ ID NO: 4, SEQ ID NO: 8, or SEQ ID NO: 12.

58. The immunohistochemistry assay of claim 50, wherein the one or more capture antibodies or one or more detection antibodies comprise:
a) a VH amino acid sequence at least 70% identical SEQ ID NO: 3 and a VL amino acid sequence at least 70% identical to SEQ ID NO: 4;
b) a VH amino acid sequence at least 70% identical to SEQ ID NO: 7 and a VL amino acid sequence at least 70% identical to SEQ ID NO: 8; or
c) a VH amino acid sequence at least 70% identical to SEQ ID NO: 11 and a VL amino acid sequence at least 70% identical to SEQ ID NO: 12.

59. The immunohistochemistry assay of claim 50, wherein the one or more detection antibodies is conjugated to colloidal gold or a cellulose nanobead.

60. (canceled)

61. The immunohistochemistry assay of claim 50, wherein one or more capture or one or more detection antibodies do not bind to non-dermatophyte organisms in the subject sample.

62-63. (canceled)

64. The immunohistochemistry assay of claim 50, wherein the one or more dermatophyte organisms are Trichophyton fungi, Microsporum fungi, or Epidermophyton fungi.

65. The immunohistochemistry assay of claim 50, wherein the one or more dermatophyte organisms are Microsporum amazonicum, Microsporum audouinii, Microsporum boullardii, Microsporum canis, Microsporum canis var. distortum, Microsporum cookei, Microsporum distortum, Microsporum duboisii, Microsporum equinum, Microsporum ferrugineum, Microsporum fulvum, Microsporum gallinae, Microsporum gypseum, Microsporum langeronii, Microsporum nanum, Microsporum persicolor, Microsporum praecox, Microsporum ripariae, Microsporum rivalieri, Epidermophyton floccosum, Epidermophyton stockdaleae, Trichophyton benhamiae, Trichophyton bullosum, Trichophyton concentricum, Trichophyton equinum, Trichophyton eriotrephon, Trichophyton erinacei, Trichophyton interdigitale, Trichophyton mentagrophytes, Trichophyton quinckeanum, Trichophyton rubrum, Trichophyton schoenleinii, Trichophyton simii, Trichophyton soudanense, Trichophyton tonsurans, Trichophyton verrucosum, and Trichophyton violaceum.

66. The immunohistochemistry assay of claim 50, wherein the one or more dermatophyte organisms are T. rubrum, T. interdigitale, E. floccosum, or M. canis.
